(19) United States
(12) Patent Application Publication
(10) Pub. No.: US 2025/0261968 A1
(43) Pub. Date: Aug. 21, 2025

(54) ARC LIFTER DISTRACTION ADJUSTOR FOR DISTRACTION HISTOGENESIS

(71) Applicant: BioDynamik, Inc., Lake Forest, CA (US)

(72) Inventors: Shanbao Cheng, Lake Forest, CA (US); Emmon Johnny Chen, Lake Forest, CA (US); Woong Kim, Seattle, WA (US)

(73) Assignee: BioDynamik, Inc., Lake Forest, CA (US)

(21) Appl. No.: 19/054,215

(22) Filed: Feb. 14, 2025

Related U.S. Application Data
(60) Provisional application No. 63/554,970, filed on Feb. 17, 2024.

(51) Int. Cl. A61B 17/66

(52) U.S. Cl. CPC A61B 17/66

(57) ABSTRACT

Systems, methods, and devices for distraction histogenesis. A device includes a rigid elongated member comprising a magnetic portion and a threaded portion. The device includes an adjustment coupler comprising a hollow interior defined by a sidewall, wherein the adjustment coupler comprises internal threading attached to the sidewall. The device includes a compliant elongated member coupled to each of the adjustment coupler and the rigid elongated member. The device is such that rotation of the rigid elongated member causes the threaded portion to screw into or out of the sidewall of the adjustment coupler.

### TECHNICAL FIELD

The disclosure relates generally to systems, methods, and devices for surgical procedures, and relates specifically to systems, methods, and devices for distraction histogenesis procedures such as periosteal distraction osteogenesis.

### BACKGROUND

In some cases, a patient benefits from perfusion improvement and tissue regeneration to aid in recovery from vascular damage, tissue trauma, bone defects, nerve damage, and other wounds. Perfusion improvement and tissue regeneration can be a major challenge for patient care and can be particularly difficult for patients experiencing chronic wounds, ischemic disease, diabetic foot ulcers, bone defects, and other health challenges.

Tissue regeneration is particularly difficult to trigger when treating diabetic patients, and especially when treating diabetic foot ulcers. Currently, there are few known successful treatments for recalcitrant diabetic foot ulcers. Ongoing research suggests there may be some success in topical therapies and oxygen treatments, but these treatments show limited success.

Additionally, it can be difficult to successfully trigger bone regeneration in response to a bone defect, fracture, or other trauma. Commonly used therapies for bone regeneration include bone graft substitutes, guided bone regeneration (GBR), distraction osteogenesis (DO), and periosteal distraction osteogenesis (PDO). PDO is a combination of tissue expansion and GBR and is implemented to create an artificial space between the bone surface and periosteum layer by expanding the periosteum, muscle, and skin at the same time.

Traditional devices for performing distraction histogenesis, and specifically for performing periosteal distraction osteogenesis, include numerous external components that may be bothersome and painful for the patient, can be difficult and time consuming to install, require numerous component implanted into patient bone tissue, and can be difficult to adjust as time goes on. What is needed are systems, methods, and devices for performing distraction histogenesis that improve patient outcomes, reduce surgery complexity, reduce the number of percutaneous sites, and reduce the quantity of cumbersome and painful external components.

In view of the foregoing, disclosed herein are systems, methods, compositions, and devices for improved surgical procedures, and specifically for improved distraction histogenesis procedures such as periosteal distraction osteogenesis.

### BRIEF DESCRIPTION OF THE DRAWINGS

Non-limiting and non-exhaustive implementations of the present disclosure are described with reference to the following figures, wherein like reference numerals refer to like parts throughout the various views unless otherwise specified. Advantages of the present disclosure will become better understood with regard to the following description and accompanying drawings where:

FIG. 1A is an overhead perspective view of a device for performing a distraction histogenesis procedure, wherein the device is in a planar configuration;

FIG. 1B is an overhead perspective view of a device for performing a distraction histogenesis procedure, wherein the device is in an expanded configuration;

FIG. 2A is an underside perspective view of a device for performing a distraction histogenesis procedure, wherein the device is in a planar configuration;

FIG. 2B is an underside perspective view of a device for performing a distraction histogenesis procedure, wherein the device is in an expanded configuration;

FIG. 3A is a straight-on side view of a device for performing a distraction histogenesis procedure, wherein the device is in a planar configuration;

FIG. 3B is a straight-on side view of a device for performing a distraction histogenesis procedure, wherein the device is in an expanded configuration;

FIG. 4A is an aerial top-down view of a device for performing a distraction histogenesis procedure, wherein the device is in a planar configuration;

FIG. 4B is an aerial top-down view of a device for performing a distraction histogenesis procedure, wherein the device is in an expanded configuration;

FIG. 5A is a straight-on underside view of a device for performing a distraction histogenesis procedure, wherein the device is in a planar configuration;

FIG. 5B is a straight-on underside view of a device for performing a distraction histogenesis procedure, wherein the device is in an expanded configuration;

FIG. 6A is a cross-sectional perspective view of a device for performing a distraction histogenesis procedure, wherein the device is in a planar configuration;

FIG. 6B is a cross-sectional perspective view of a device for performing a distraction histogenesis procedure, wherein the device is in a distraction configuration;

FIG. 7A is an overhead perspective view of a device for performing a distraction histogenesis procedure, wherein the device is in a planar configuration;

FIG. 7B is an overhead perspective view of a device for performing a distraction histogenesis procedure, wherein the device is in an expanded configuration;

FIG. 8A is an underside perspective view of a device for performing a distraction histogenesis procedure, wherein the device is in a planar configuration;

FIG. 8B is an underside perspective view of a device for performing a distraction histogenesis procedure, wherein the device is in an expanded configuration;

FIG. 9A is a straight-on side view of a device for performing a distraction histogenesis procedure, wherein the device is in a planar configuration;

FIG. 9B is a straight-on side view of a device for performing a distraction histogenesis procedure, wherein the device is in an expanded configuration;

FIG. 10A is a cross-sectional perspective view of a device for performing a distraction histogenesis procedure, wherein the device is in a planar configuration;

FIG. 10B is a cross-sectional perspective view of a device for performing a distraction histogenesis procedure, wherein the device is in a distraction configuration;

FIG. 11A is an overhead perspective view of a device for performing a distraction histogenesis procedure, wherein the device is in a planar configuration;

FIG. 11B is an overhead perspective view of a device for performing a distraction histogenesis procedure, wherein the device is in an expanded configuration;

FIG. 12A is an underside perspective view of a device for performing a distraction histogenesis procedure, wherein the device is in a planar configuration;

FIG. 12B is an underside perspective view of a device for performing a distraction histogenesis procedure, wherein the device is in an expanded configuration;

FIG. 13A is a straight-on side view of a device for performing a distraction histogenesis procedure, wherein the device is in a planar configuration;

FIG. 13B is a straight-on side view of a device for performing a distraction histogenesis procedure, wherein the device is in an expanded configuration;

FIG. 14A is an aerial top-down view of a device for performing a distraction histogenesis procedure, wherein the device is in a planar configuration;

FIG. 14B is an aerial top-down view of a device for performing a distraction histogenesis procedure, wherein the device is in an expanded configuration;

FIG. 15 is a cross-sectional perspective view of a device for performing a distraction histogenesis procedure, wherein the device is in a planar configuration;

FIG. 16 is a cross-sectional perspective view of a device for performing a distraction histogenesis procedure, wherein the device is in a distraction configuration;

FIG. 17 is a schematic illustration a system for performing a distraction histogenesis procedure;

FIG. 18A is a schematic illustration of magnetic coupling between an external magnetic controller and a device for performing a distraction histogenesis procedure, wherein the device is in a planar configuration;

FIG. 18B is a schematic illustration of magnetic coupling between an external magnetic controller and a device for performing a distraction histogenesis procedure, wherein the device is in an expanded configuration;

FIG. 19A is a schematic aerial top-down view of an external magnetic controller;

FIG. 19B is a schematic cross-sectional side view of an external magnetic controller that includes an external magnet;

FIG. 19C is a schematic cross-sectional side view of an external magnetic controller that includes an external magnet that may be driven by a motor;

FIG. 20A is a schematic cross-sectional side view of an external magnetic controller that includes an external magnet that may be driven by an electromagnet;

FIG. 20B is a schematic cross-sectional side view of an external magnetic controller, wherein the external magnetic controller includes an electromagnet;

FIGS. 21A-21F are schematic illustrations of a system for driving an external magnet of an external magnetic controller with a two-pole electromagnet;

FIG. 22A is a schematic illustration of a magnetic coupling between an external magnetic controller and an implant magnet of an arc lifter distraction device;

FIG. 22B is a schematic illustration of a magnetic coupling between an external magnetic controller and an implant magnet of an arc lifter distraction device;

FIG. 23A is a schematic illustration of a cross-sectional aerial top-down view of a magnetic portion of a rigid elongated member of a magnetically-actuated arc lifter distraction device, or an external magnet of an EMC that includes two poles;

FIG. 23B is a schematic illustration of a cross-sectional aerial top-down view of a magnetic portion of a rigid elongated member of a magnetically-actuated arc lifter distraction device, or an external magnet of an EMC that includes two poles;

FIG. 23C is a schematic illustration of a cross-sectional aerial top-down view of a magnetic portion of a rigid elongated member of a magnetically-actuated arc lifter distraction device, or an external magnet of an EMC that includes two poles;

FIG. 24A is a schematic straight-on side view of an elevator for use in connection with a distraction surgical procedure;

FIG. 24B is a schematic aerial top-down view of an elevator for use in connection with a distraction surgical procedure;

FIG. 25 is a schematic illustration of a system for manually adjusting an arc lifter distraction device;

FIG. 26A is a schematic cross-sectional illustration of a surgical positioning of an arc lifter distraction device, wherein the device is in a planar configuration;

FIG. 26B is a schematic cross-sectional illustration of a surgical positioning of an arc lifter distraction device, wherein the device is in an expanded configuration;

FIGS. 27A-27C are schematic block diagrams of a method for performing a distraction surgical procedure with an arc lifter distraction device;

FIGS. 28A-28C are schematic block diagrams of a method for performing a distraction surgical procedure with a magnetically actuated arc lifter distraction device;

FIG. 29 is a schematic block diagram of a system for monitoring health data and remotely actuating adjustment of a distraction device; and

FIG. 30 is a schematic block diagram of an example computing device.

### DETAILED DESCRIPTION

Described herein are systems, methods, and devices for triggering perfusion improvement, growth factors releasement, and tissue regeneration. The disclosures herein may be implemented to trigger regeneration of bone tissue, vascular tissue, microvascular tissue, skin tissue, nerve tissue, and other tissue types. The systems, methods, and devices described herein may be utilized to perform distraction histogenesis surgical procedures on various tissue types and may specifically be utilized to perform periosteal distraction procedures and other distraction histogenesis procedures to trigger regeneration of soft tissues.

A device described herein is designed for performing a distraction histogenesis surgical procedure on a patient. The device may be referred to as a distraction device, arc lifter, or arc lifter distraction device as described herein. The device includes a rigid elongated member and a compliant elongated member that is coupled to the rigid elongated member. The compliant elongated member is designed to bend away from a longitudinal axis of the rigid elongated member in response to adjusting a distance between opposing ends of the compliant elongated member. The distance between the opposing ends of the compliant elongated member may be adjusted by, for example, moving one end of the compliant elongated member nearer to the opposite end.

The distraction devices described herein may be designed for magnetic actuation or driver-based actuation. The magnetic actuation distraction devices described herein include a magnetic portion designed to form a strong magnetic coupling with a corresponding magnet on an external magnetic controller. The external magnetic controller may cause rotation of the magnetic portion, which may in turn cause distraction or retraction of a compliant elongated member. The driver-based actuation distraction devices described herein may include a means to rotate the rigid elongated member with a screwdriver or other similar device. The rotation of the rigid elongated member may in turn cause distraction or retraction of a compliant elongated member.

The distraction devices described herein are designed to be installed in between patient tissue layers such that bending the compliant elongated member causes distraction of a top tissue layer relative to a bottom tissue layer. Likewise, relaxing a bend in the compliant elongated member causes retraction of the top tissue layer relative to the bottom tissue layer. The distraction devices described herein may be utilized to perform periosteal distraction surgical procedures and may further be utilized to perform distraction histogenesis procedures on other tissue types, including, for example, vascular tissue, microvascular tissue, skin tissue, nerve tissue, and so forth.

Periosteal distraction is a surgical technique that is traditionally used to treat bone defects by stimulating bone growth. Periosteal distraction is designed to gradually stretch the periosteum away from the underlying bone tissue, which may additionally separate the periosteum from the underlying bone tissue. This may create an artificial space between the bone surface and periosteum. In response, the body generates new bone tissue by gradually expanding the periosteum with no need for corticotomy. Periosteal distraction is minimally invasive when compared to traditional bone grafting techniques, and the procedure encourages the body to generate its own bone, which typically integrates more successfully than synthetic materials.

What is needed are improved systems, methods, and devices for periosteal distraction that reduce the risks of infections, reduce the risks of device complications, reduce surgery complexity, reduce the quantity of percutaneous sites prone to infection, reduce the number of external components that may be cumbersome and painful for the patient, and reduce the complexity of tasks performed after surgery. Additionally, what is needed are systems, methods, and devices for triggering regeneration of non-bone tissue types through distraction histogenesis, including, for example, promoting revascularization for increasing perfusion to promote the healing of diabetic foot ulcers and other chronic wounds.

In view of the foregoing, described herein systems, methods, and devices for initiating tissue growth through distraction histogenesis. The distraction histogenesis techniques described herein may be utilized to treat tissue defects by raising systemic growth factor levels. One or more of the devices described herein can be coupled to many different tissues in the body, because the devices are internal and implantable.

In the following description of the disclosure, reference is made to the accompanying drawings, which form a part hereof, and in which is shown by way of illustration specific implementations in which the disclosure may be practiced. It is understood that other implementations may be utilized, and structural changes may be made without departing from the scope of the disclosure.

Before the structures, systems, methods, and compositions described herein are disclosed, it is to be understood that this disclosure is not limited to the particular structures, configurations, process steps, and materials disclosed herein as such structures, configurations, process steps, and materials may vary somewhat. It is also to be understood that the terminology employed herein is used for the purpose of describing particular embodiments only and is not intended to be limiting since the scope of the disclosure will be limited only by the appended claims and equivalents thereof.

In describing and claiming the subject matter of the disclosure, the following terminology will be used in accordance with the definitions set out below.

As used herein, the terms “comprising,” “including,” “containing,” “characterized by,” and grammatical equivalents thereof are inclusive or open-ended terms that do not exclude additional, unrecited elements or method steps.

As used herein, the phrase “consisting of” and grammatical equivalents thereof exclude any element, step, or ingredient not specified in the claim.

As used herein, the phrase “consisting essentially of” and grammatical equivalents thereof limit the scope of a claim to the specified ingredients, materials, or steps and those that do not materially affect the basic and novel characteristic or characteristics of the claimed disclosure.

Unless defined otherwise, all technical and scientific terms used herein have the same meanings as commonly understood by one of ordinary skill in the art to which this disclosure pertains and belongs.

Reference will now be made in detail to the exemplary embodiments, examples of which are illustrated in the accompanying drawings. Wherever possible, the same reference numbers are used throughout the drawings to refer to the same or like parts. It is further noted that elements disclosed with respect to particular embodiments are not restricted to only those embodiments in which they are described. For example, an element described in reference to one embodiment or figure, may be alternatively included in another embodiment or figure regardless of whether or not those elements are shown or described in another embodiment or figure. In other words, elements in the figures may be interchangeable between various embodiments disclosed herein, whether shown or not.

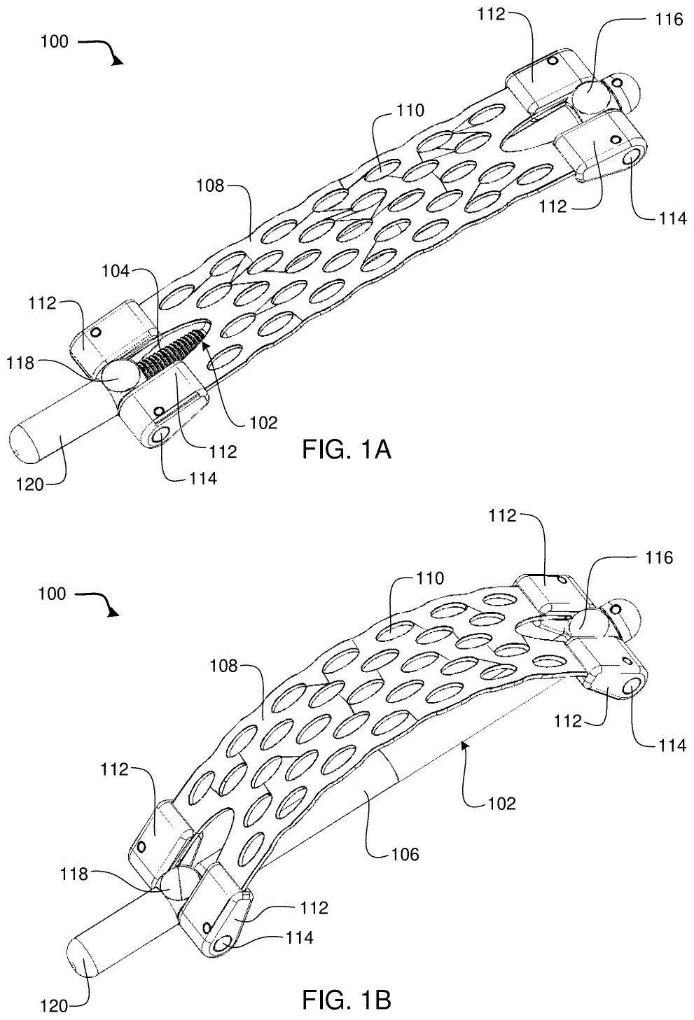

Referring now to the figures, FIGS. 1A and 1B are overhead perspective views of a device 100 for performing a distraction histogenesis surgical procedure. FIG. 1A illustrates wherein the device 100 is in a planar state, and FIG. 1B illustrates wherein the device 100 is in an expanded state. The device 100 may specifically be utilized to perform a periosteal distraction osteogenesis procedure to lift a periosteum tissue layer away from a bone tissue. The device 100 may be utilized for other distraction histogenesis procedures, including those performed on soft tissues.

The device 100 is configured to be implanted within a patient during a surgical procedure. After implantation, the device 100 may be distracted or retracted according to the patient's needs. In an example use-case, the device 100 is implanted in between bone tissue and a periosteum layer of a patient. The surgical site may be fully closed over the device 100 to prevent infection, and this may include suturing shut one or more of the periosteum or skin layers over the device 100. The device 100 may then be magnetically actuated without physically touching the device. The magnetic coupling may be performed with an external magnetic controller (EMC) as described herein, or another similar device. A user may thereby utilize an external magnet or electromagnet to distract or retract the device 100 in a controlled manner while the device 100 is installed in the patient.

The device 100 includes a rigid elongated member 102, which is only partially visible in the views in FIGS. 1A-1B. The rigid elongated member 102 may include a threaded portion 104 attached to a magnetic portion 106. The device 100 may include a cap 120 configured to receive and encase the threaded portion 104 of the rigid elongated member 102. The threaded portion 104 is visible in FIG. 1A because the device 100 is fully planar, and the rigid elongated member 102 comprises its maximum length. The threaded portion 104 is not visible in FIG. 1B because the device 100 is fully distracted, and the threaded portion 104 is fully disposed within the cap 120.

The threaded portion 104 of the rigid elongated member 102 comprises threading the corresponds with internal threading (not visible) disposed within an adjustment coupler 118. The rigid elongated member 102 is rotatable about its longitudinal axis (i.e., a “roll” rotation). When the rigid elongated member 102 rotates about its longitudinal axis, the threaded portion 104 screws into and out of the adjustment coupler 118.

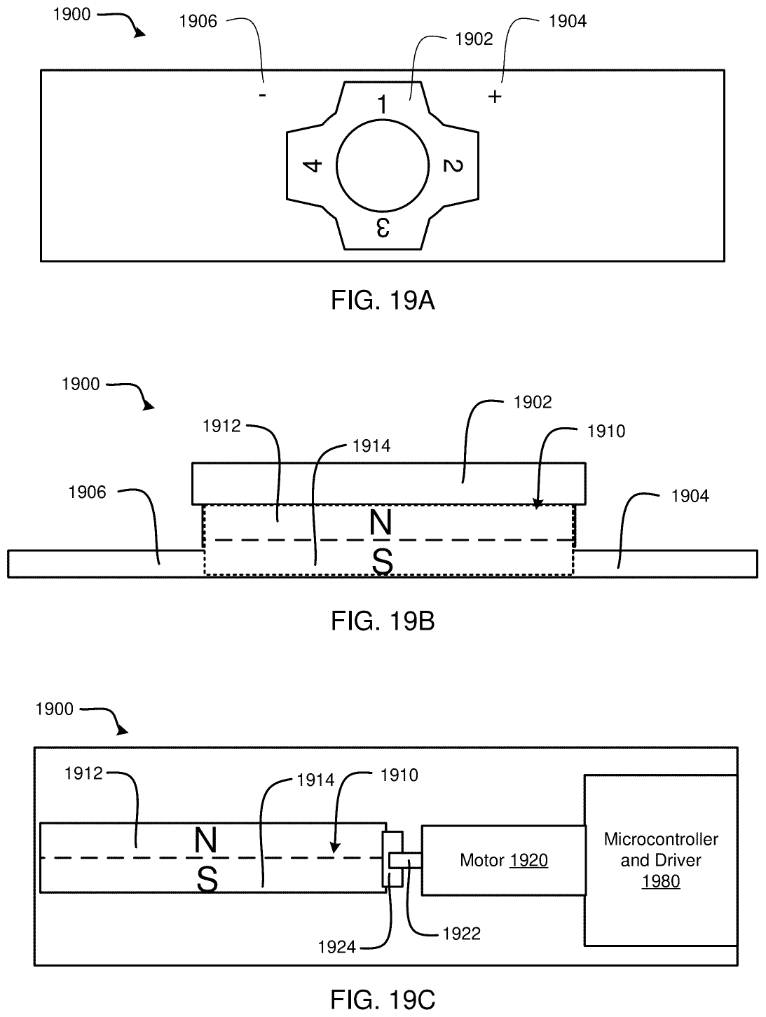

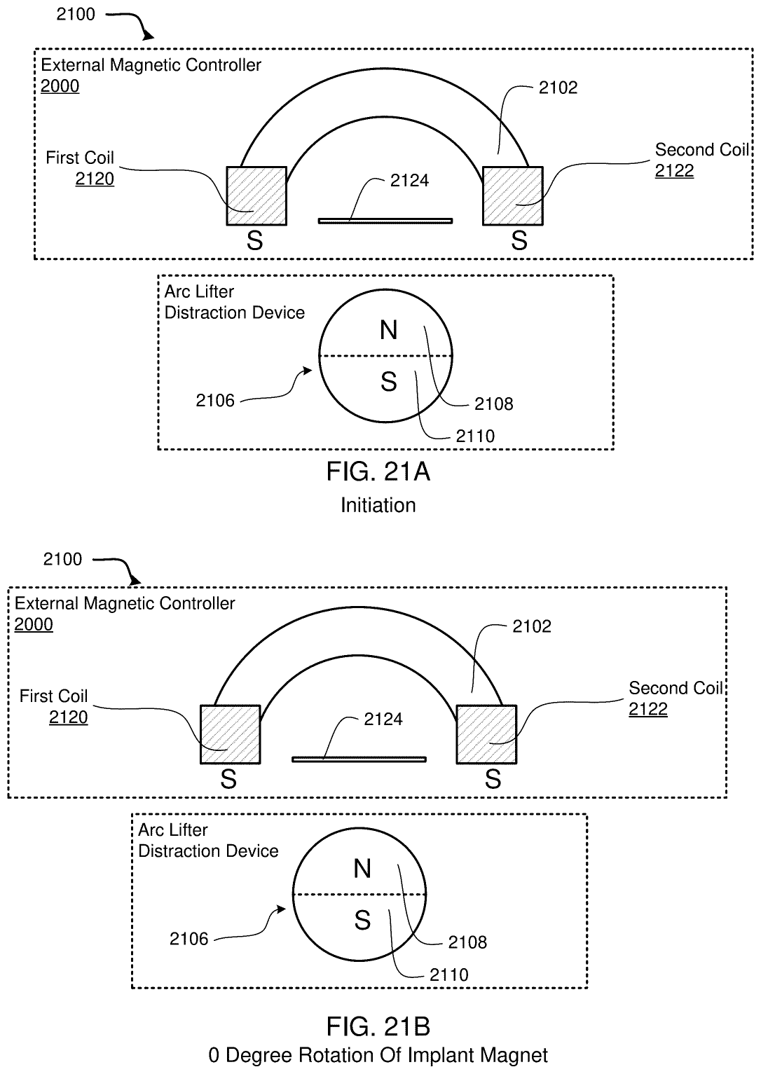

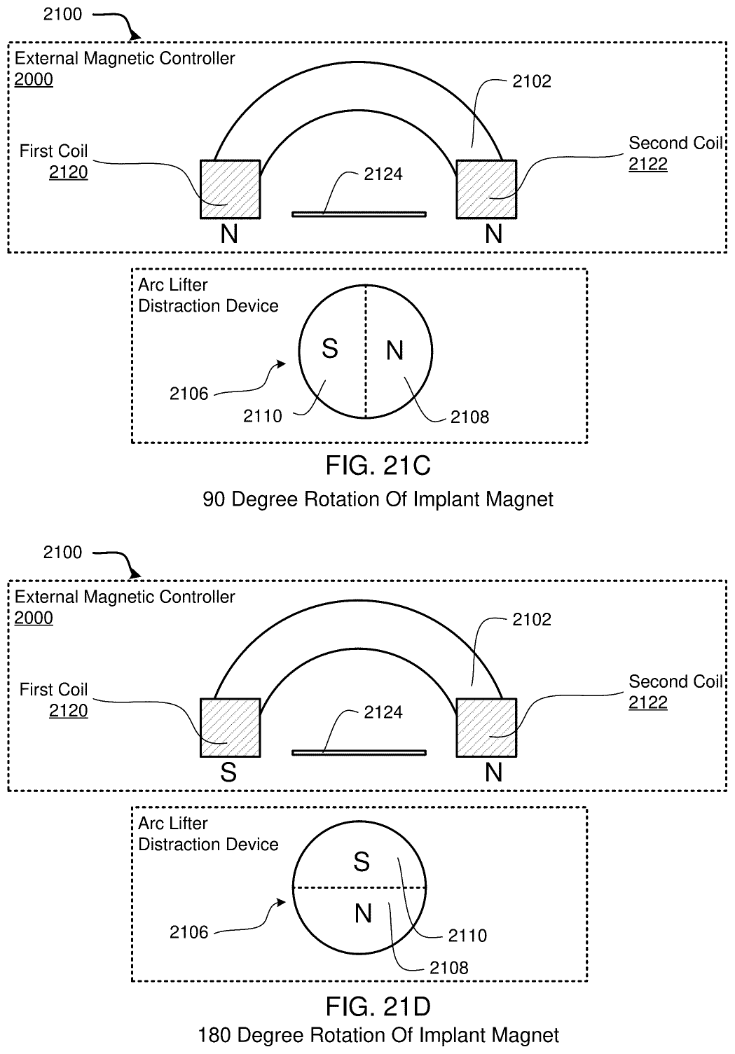

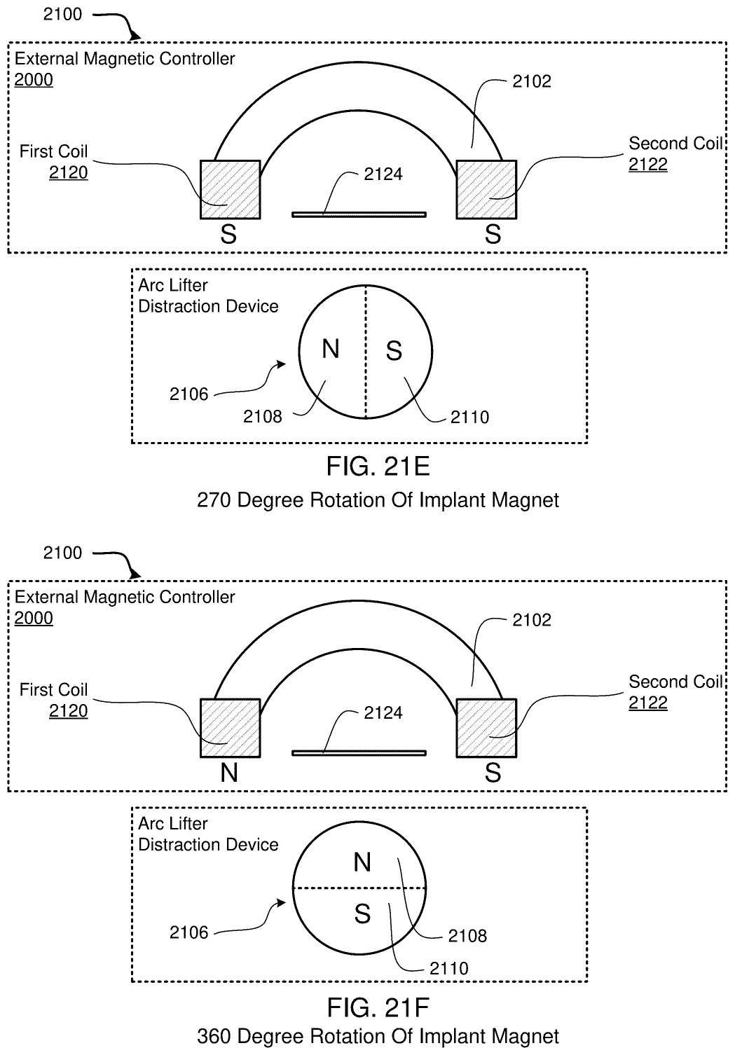

When the device 100 is installed within a patient, the rigid elongated member 102 is rotated about its longitudinal axis after establishing a magnetic coupling between the magnetic portion 106 of the rigid elongated member 102, and a corresponding EMC (see e.g., EMC 1900 first illustrated at FIG. 19 and/or EMC 2000 first illustrated at FIG. 21) or similar device. The magnetic portion 106 includes an elongated magnet comprising two or more poles each running a length of the elongated magnet. The EMC includes a corresponding elongated magnet that forms a magnetic coupling with the magnetic portion 106. When the corresponding elongated magnet of the EMC is rotated, this causes the magnetic portion 106 to rotate, and thereby causes the rigid elongated member 102 to rotate.

The device 100 includes a compliant elongated member 108 that is coupled to the adjustment coupler 118 at a first end and is additionally coupled to a stationary coupler 116 at an opposite end. The compliant elongated member 108 is capable of bending away from the longitudinal axis of the rigid elongated member 102. In the view illustrated in FIG. 1B, the compliant elongated member 108 is bending away from the rigid elongated member 102 to form an arch configuration.

The compliant elongated member 108 is coupled to the stationary coupler 116 and the adjustment coupler 118 by way of one or more pivot heads 112. As shown in FIGS. 1A and 1B, the compliant elongated member 108 may be coupled to the stationary coupler 116 by way of two pivot heads 112 that are located on either side of the stationary coupler 116. Likewise, the compliant elongated member 108 may be coupled to the adjustment coupler 118 by way of two pivot heads 112 that are located on either side of the adjustment coupler 118. Each pivot head 112 is attached to its corresponding coupler 116, 118 by way of a pivot fastener 114. The pivot fastener 114 enables the pivot head 112 to rotate about the center of the pivot fastener 114. The pivot fastener 114 may include, for example, a bearing bolt, axle screw, pivot bolt, rotating bolt, spindle bolt, turning screw, and so forth.

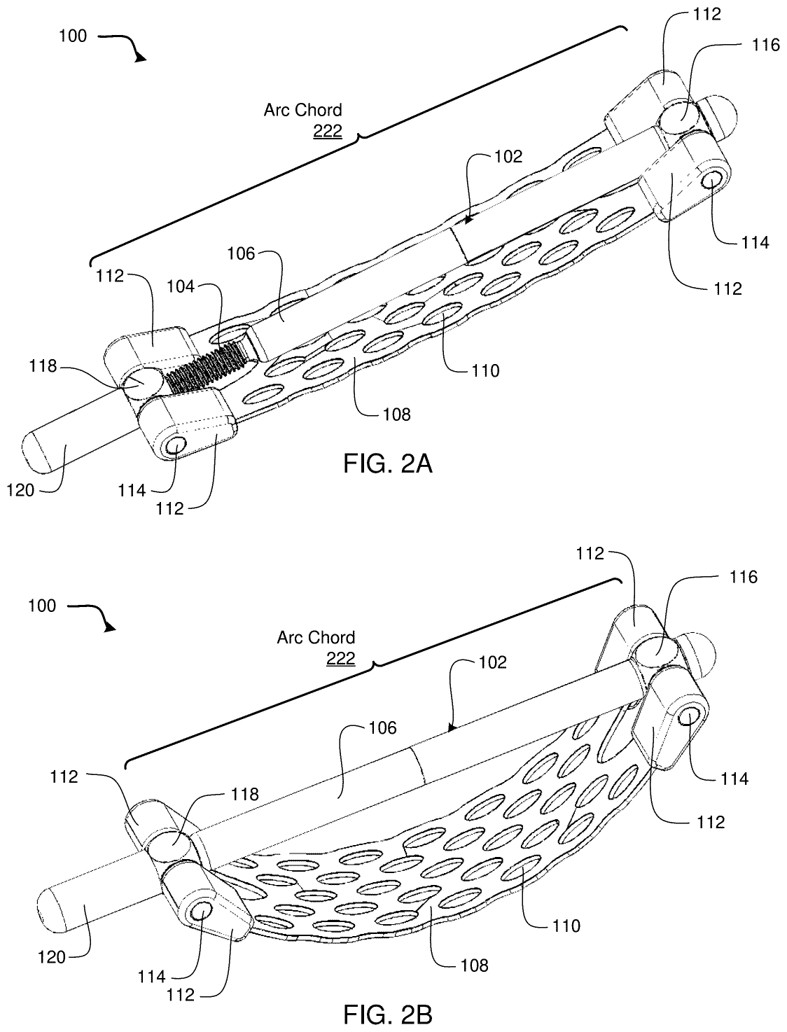

The compliant elongated member 108 may include a plurality of holes 110 disposed therethrough. The plurality of holes 110 may serve to reduce the overall mass of the device 100. The holes 110 may additionally allow for fluids and tissues to pass through the compliant elongated member 108, and in some cases, this may improve patient outcomes. Additionally, the holes 110 may improve the flexibility of the compliant elongated member 108 to ensure it can form a smooth arch when the device 100 is distracted as shown in FIG. 1B. FIGS. 2A and 2B are underside perspective view of the device 100 for performing a distraction histogenesis procedure, wherein FIG. 2A illustrates the device 100 in a planar state, and FIG. 2B illustrates the device 100 in an expanded state.

FIGS. 2A and 2B bring attention to what may be referred to herein as the arc chord 222, which is a distance between opposite end points of the compliant elongated member 108. The arc chord 222 may be measured from the pivot fastener 114 of the adjustment coupler 118 to the pivot fastener 114 of the stationary coupler 116 as shown in FIGS. 2A-2B. However, the arc chord 222 may be measured from other endpoints as deemed appropriate. The arc chord 222 refers to the straight point-to-point distance of the arc formed by the compliant elongated member 108. When the device 100 is in a planar state as shown in FIG. 2A, the arc chord 222 comprises a longer length. When the device 100 is in an expanded state as shown in FIG. 2B, the arc chord 222 comprises a shorter length.

As shown in FIGS. 2A and 2B, the threaded portion 104 of the device 100 is exposed when the device is in a planar state and the arc chord 222 comprises its maximum length. The rigid elongated member 102 may be rotated about its longitudinal axis (i.e., a “roll” rotation) in response to a corresponding elongated magnet of an EMC causing rotation of the magnetic portion 106. When the EMC causes the magnetic portion 106 to rotate in a tightening direction, the threaded portion 104 will screw further into the internal threading (not visible) disposed within the adjustment coupler 118. When the EMC causes the magnetic portion 106 to rotate in a loosening direction, the threaded portion 104 unscrews from the internal threading (not visible) disposed within the adjustment coupler 118. Thus, an EMC may be actuated to adjust the total length of the arc chord 222, and to thereby cause adjustment in the arc height of the compliant elongated member 108.

FIG. 2B illustrates wherein the total length of the arc chord 222 has been shortened by screwing the threaded portion 104 through the corresponding internal threading (not visible) disposed within the adjustment coupler 118. The threaded portion 104 is then disposed at least partially within the cap 120, and the compliant elongated member 108 arches further away from the rigid elongated member 102. The cap 120 may serve to protect a patient from sustaining tissue damage from the threaded portion 104 after the threaded portion 104 has been screwed through the corresponding internal threading of the adjustment coupler 118.

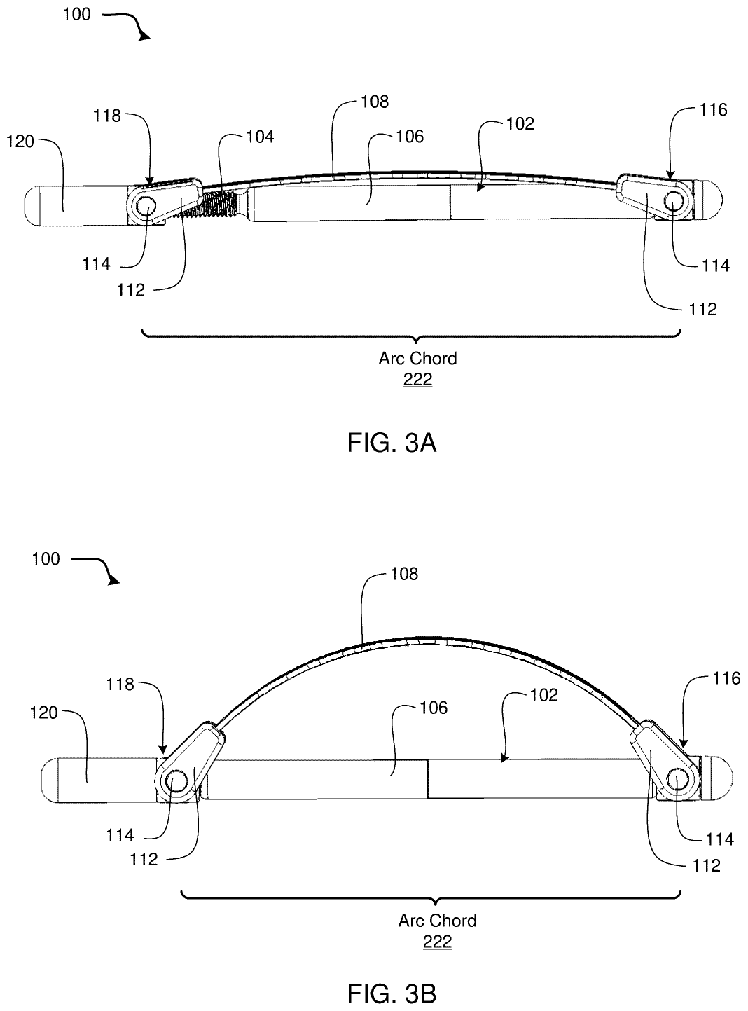

FIGS. 3A and 3B are straight-on side views of the device 100 for performing a distraction histogenesis procedure, wherein FIG. 3A illustrates the device 100 in a planar state, and FIG. 3B illustrates the device 100 in an expanded state.

FIGS. 3A and 3B illustrate rotation of the pivot heads 112 about the pivot fasteners 114. When the total length of the arc chord 222 is shortened by screwing the threaded portion 104 through the adjustment coupler 118 and into the cap 120, the compliant elongated member 108 will arch away from the rigid elongated member 102 as shown in FIG. 3B.

The length of the compliant elongated member 108 may be optimized such that the compliant elongated member 108 arches slightly away from the rigid elongated member 102 even when the rigid elongated member 102 is at its maximum length, as shown in FIG. 3A. Thus, the compliant elongated member 108 may comprise a length that is slightly longer than the distance between the coupling points of the stationary coupler 116 and the adjustment coupler 118. This may be desirable to ensure the compliant elongated member 108 arches in the desired direction when the device 100 is distracted as shown in FIG. 3B.

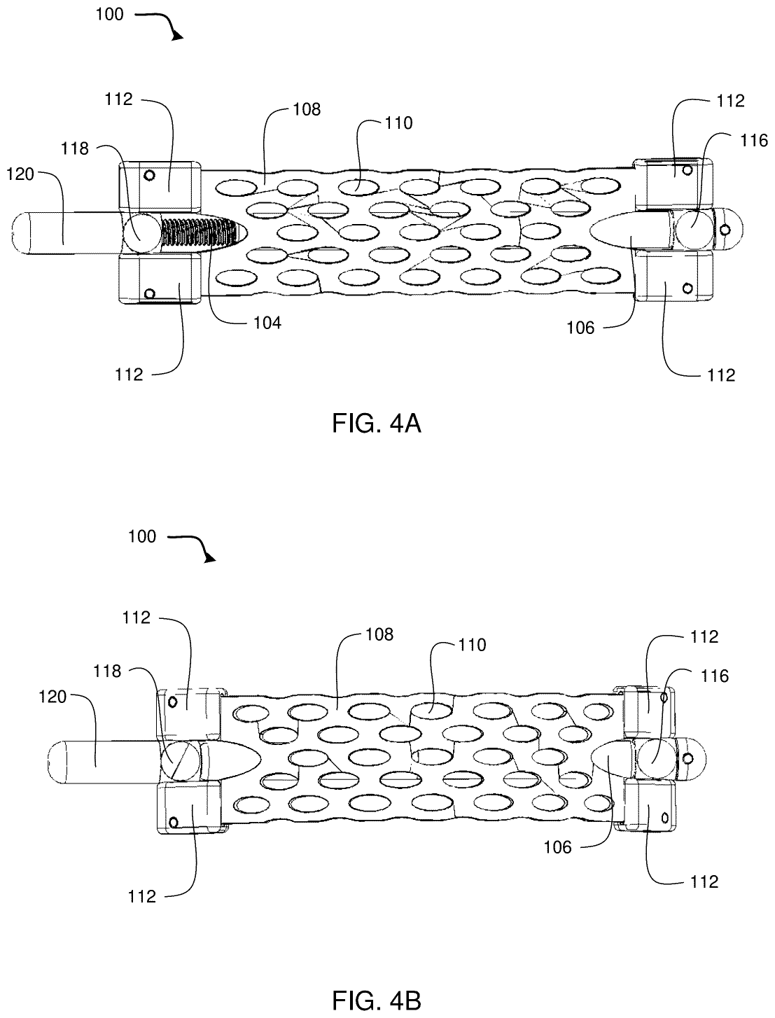

FIGS. 4A and 4B are top-down aerial views of the device 100 for performing a distraction histogenesis procedure, wherein FIG. 4A illustrates the device 100 in a planar state, and FIG. 4B illustrates the device 100 in an expanded state.

The compliant elongated member 108 may comprise the plurality of holes 110 disposed therethrough, as shown in FIGS. 4A and 4B. In alternative implementations, the compliant elongated member 108 may include no holes 110 or may include a different quantity or arrangement of holes 110. The compliant elongated member 108 is constructed of a material that is sufficiently flexible to allow for the material to bend when the device 100 is distracted, but rigid enough to maintain its shape and prevent collapse when the compliant elongated member 108 presses against patient tissues. The compliant elongated member 108 may specifically be constructed of one or more an elastic alloy, a super elastic polymer, an elastic composite, stainless steel, a titanium polymer, a combination of polymers over metal element to allow for drug elution capabilities, and so forth. The compliant elongated member 108 may specifically be constructed of a super elastic Nitinol sheet or other super elastic alloy.

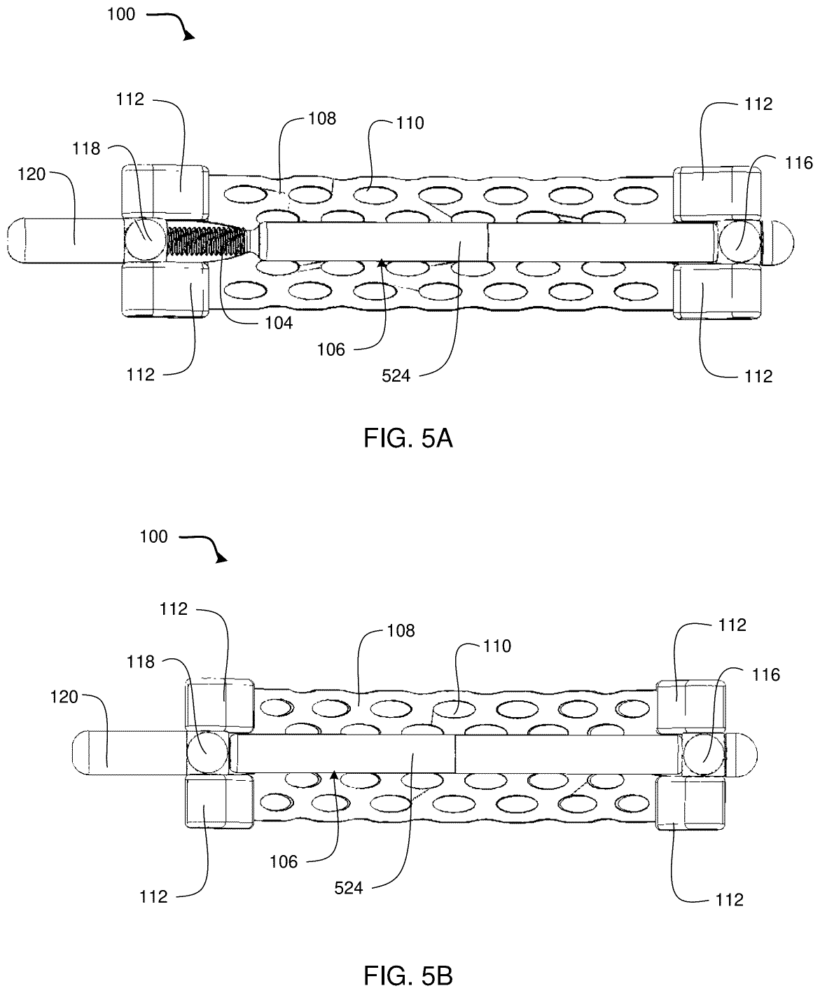

FIGS. 5A and 5B are underside views of the device 100 for performing a distraction histogenesis procedure, wherein FIG. 5A illustrates the device 100 in a planar state, and FIG. 5B illustrates the device 100 in an expanded state.

The magnetic portion 106 of the rigid elongated member 102 may include an elongated magnet that is disposed within a housing 524. The housing 524 may include a hollow cylindrical sheath configured to receive the elongated magnet. The housing 524 may be constructed of a material that will cause minimal disruption to the magnetic coupling between the elongated magnet of the magnetic portion 106, and the corresponding elongated magnet of an EMC. Such housing material is usually nonferrous metal or plastic.

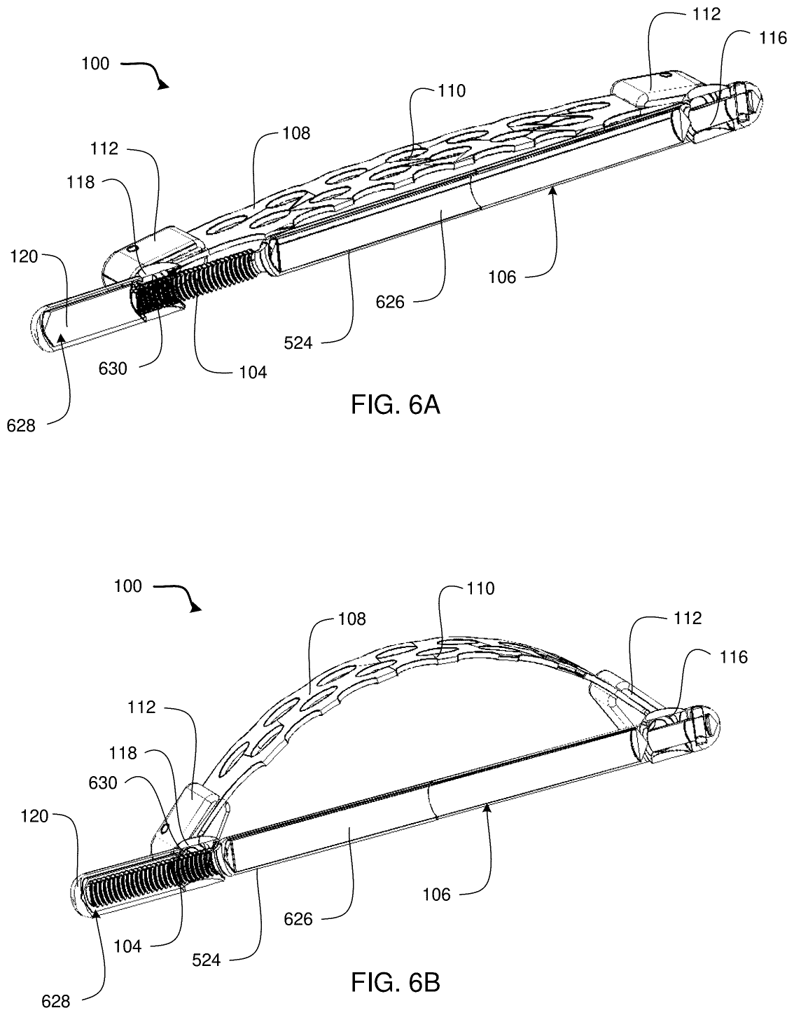

FIGS. 6A and 6B are perspective cross-sectional views of the device 100 for performing a distraction histogenesis procedure, wherein FIG. 6A illustrates the device 100 in a planar state, and FIG. 6B illustrates the device 100 in an expanded state.

The cross-sectional views illustrated in FIGS. 6A and 6B enable visualization of the internal threading 630 of the adjustment coupler 118. The adjustment coupler 118 may comprise a hollow cylindrical geometry defined by a sidewall and may include the internal threading 630 attached to an interior side of the sidewall. The internal threading 630 of the adjustment coupler 118 corresponds with the external threading of the threaded portion 104 of the rigid elongated member 102. The cross-sectional views further enable visualization of the hollow interior 628 defined by the cap 120. The hollow interior 628 is configured to receive and encase the threaded portion 104 when the device 100 is distracted as shown in FIG. 6B.

The cross-sectional views further enable visualization of components of the magnetic portion 106, including an elongated magnet 626 disposed within a housing 524. The elongated magnet 626 may run an entire length of the housing 524 or may include a length shorter than the housing 524. The elongated magnet 626 may include two or more diametrically magnetized magnetic poles that run an entire length or shorter of the elongated magnet 626.

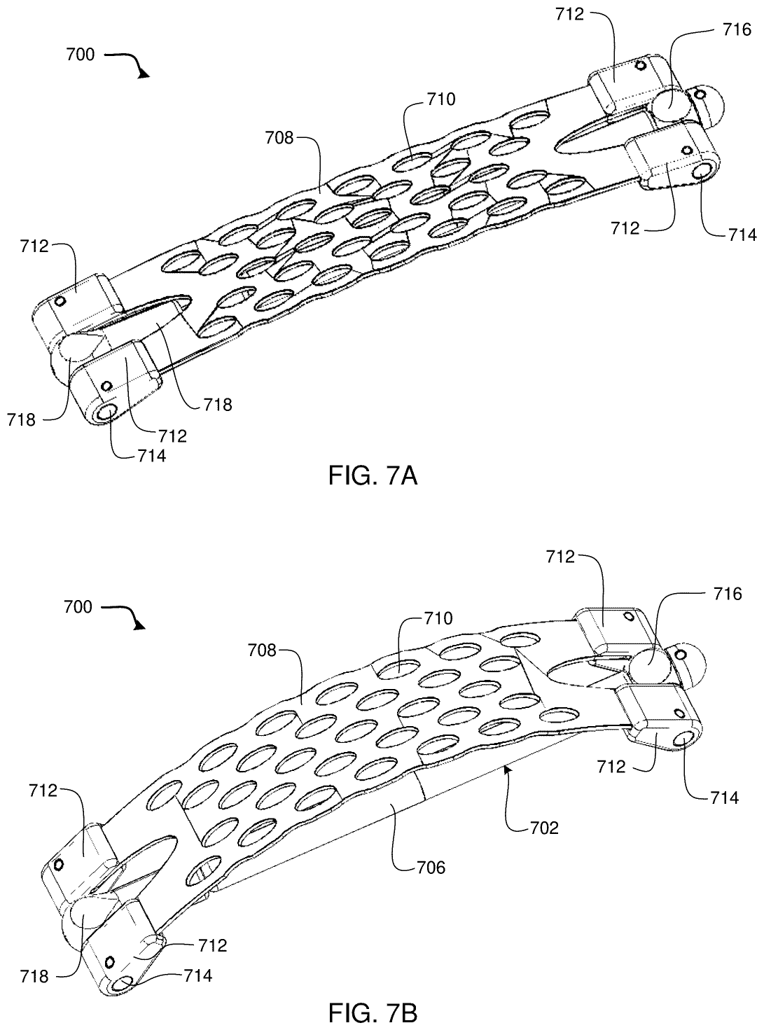

FIGS. 7A and 7B are overhead perspective views of a device 700 for performing a distraction histogenesis procedure. FIG. 7A illustrates wherein the device 700 is in a planar state, and FIG. 7B illustrates wherein the device 700 is in an expanded state. The device 700 may specifically be utilized to perform a periosteal distraction procedure to lift a periosteum tissue layer away from a bone tissue. The device 700 may be utilized for other distraction histogenesis procedures, including those performed on soft tissues.

The device 700 is similar to the device 100 first described in connection with FIGS. 1A-1B, but with some implementational differences. For example, the device 700 does not include a cap (see 120) that protrudes outward relative to an adjustment coupler (see 118). Additionally, the device 700 does not include exposed external threading like the threaded portion (see 104) of the device 100 first described in connection with FIGS. 1A-1B.

The device 700 includes a rigid elongated member 702, which is only partially visible in the views of FIGS. 7A-7B. The rigid elongated member 702 includes a magnetic portion 706 and a threaded portion (not visible in FIGS. 7A-7B) that is attached directly to the magnetic portion 706. The threaded portion of the rigid elongated member 702 comprises external threading that corresponds with internal threading (not visible) disposed within an adjustment coupler 718.

The device 700 includes a compliant elongated member 708 that may optionally include a plurality of holes 710 disposed therethrough. The compliant elongated member 708 is coupled to each of a stationary coupler 716 and the adjustment coupler 718 by way of a plurality of pivot heads 712 as shown. Each of the pivot heads 712 includes a pivot fastener 714 enabling the pivot head 712 to rotate and enable the compliant elongated member 708 to bend away from the rigid elongated member 702 as shown at least in FIG. 7B.

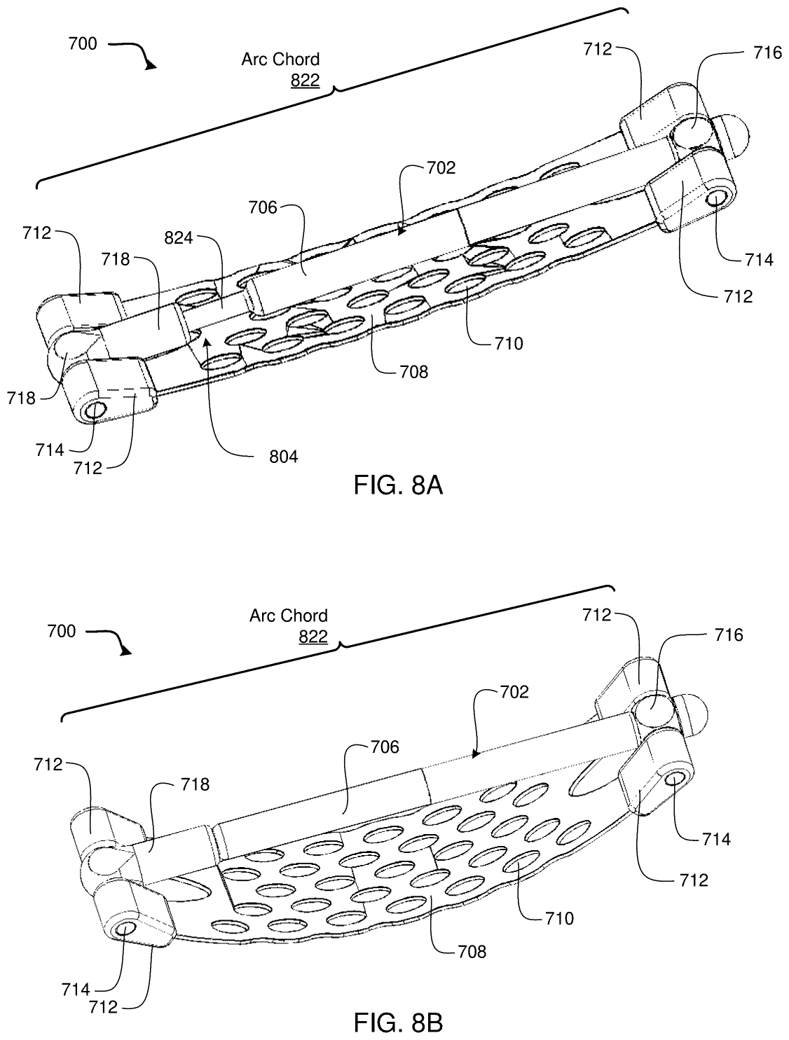

FIGS. 8A and 8B are underside perspective views of the device 700 for performing a distraction histogenesis procedure, wherein FIG. 8A illustrates the device 700 in a planar state, and FIG. 8B illustrates the device 700 in an expanded state.

FIGS. 8A and 8B bring attention to what may be referred to the arc chord 822, which is a distance between opposite end points of the compliant elongated member 708. The arc chord 822 may be measured from the pivot fastener 714 of the adjustment coupler 718 to the pivot fastener 714 of the stationary coupler 716 as shown in FIGS. 8A-8B. However, the arc chord 822 may be measured from other endpoints as deemed appropriate. The arc chord 822 refers to the straight point-to-point distance of the arc formed by the compliant elongated member 708. When the device 700 is in a planar state as shown in FIG. 8A, the arc chord 822 comprises a longer length. When the device 700 is in an expanded state as shown in FIG. 8B, the arc chord 822 comprises a shorter length.

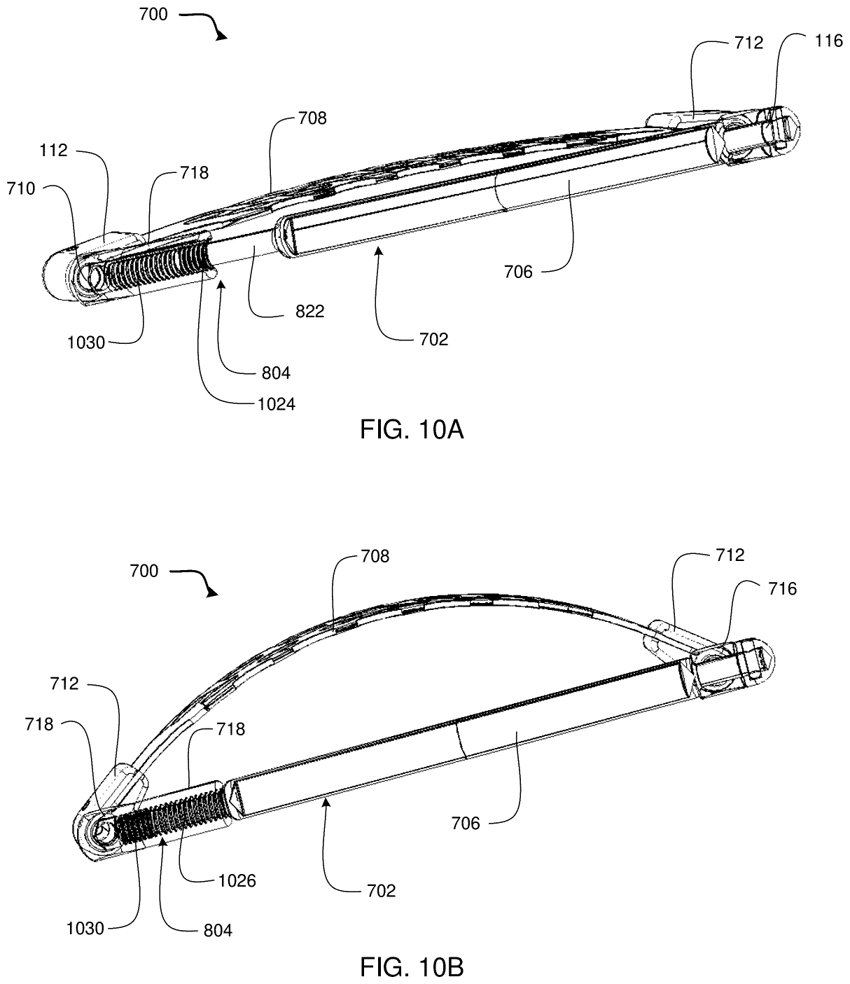

The underside views illustrated in FIGS. 8A-8B enable viewing of a shank 824 of the threaded portion 804 of the rigid elongated member 702. The threaded portion 804 additionally includes external threading (see 1024 at FIGS. 10A-10B) that begins adjacent to the shank 824 and is visible in only the cross-sectional views of FIGS. 10-10B. The rigid elongated member 702 is rotatable about its longitudinal axis (i.e., a “roll” rotation). When the rigid elongated member 702 rotates about its longitudinal axis, the external threading (see 1024) of the threaded portion 804 screws into and out of the adjustment coupler 718. The total length of the arc chord 824 is determined based upon where the threaded portion 804 is located relative to the adjustment coupler 718.

When the device 700 is installed within a patient, the rigid elongated member 702 is rotated about its longitudinal axis after establishing a magnetic coupling between the magnetic portion 706 of the rigid elongated member 702, and a corresponding EMC or similar device. The magnetic portion 706 includes an elongated magnet comprising two or more poles each running a length of the elongated magnet. The EMC includes a corresponding elongated magnet that forms a magnetic coupling with the magnetic portion 706. When the corresponding elongated magnet of the EMC is rotated, this causes the magnetic portion 706 to rotate, and thereby causes the rigid elongated member 702 to rotate. This further causes the external threading (see 1024) of the threaded portion 804 to screw into or out of corresponding internal threading disposed within the adjustment coupler 718.

When the threaded portion 804 is fully screwed into the adjustment coupler 718, the arc chord 822 comprises a shortened total length, and the compliant elongated member 708 bends away from the rigid elongated member 702 as shown in FIG. 8B. Conversely, when the threaded portion 804 is unscrewed from the adjustment coupler 718, the arc chord 822 comprises a longer total length, and the compliant elongated member 708 may comprise a straight configuration or may slightly bend away from the rigid elongated member 702 as shown in FIG. 8A.

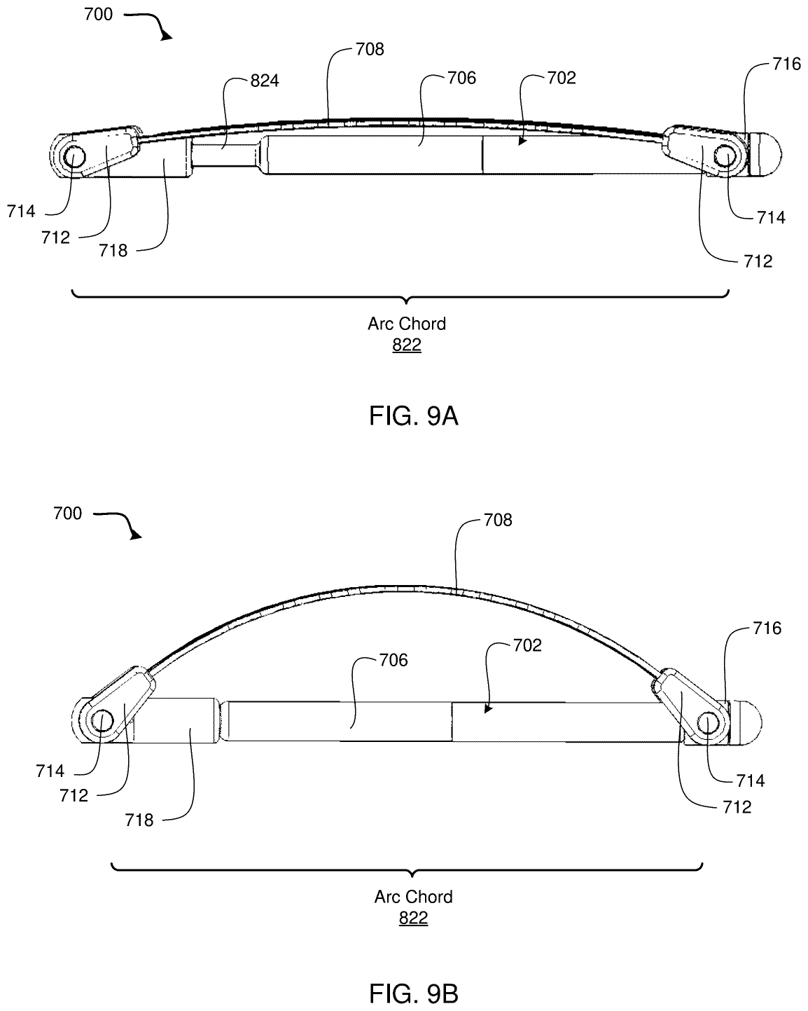

FIGS. 9A and 9B are straight-on side views of the device 700 for performing a distraction histogenesis procedure, wherein FIG. 9A illustrates the device 700 in a planar state, and FIG. 9B illustrates the device 700 in an expanded state.

The device 700 may include a stopper to prevent the rigid elongated member 702 from being fully unscrewed from the adjustment coupler 718. This stopper may ensure the rigid elongated member 702 remains coupled to each of the adjustment coupler 718 and the stationary coupler 716 when the device 700 is installed in a patient. The stopper may ensure that the configuration illustrated in FIG. 9A represents a fully planar state, wherein the rigid elongated member 702 is unscrewed from the adjustment coupler 718 as much as possible, and the rigid elongated member 702 therefore has its maximum total length.

FIGS. 10A and 10B are perspective cross-sectional views of the device 700 for performing a distraction histogenesis procedure, wherein FIG. 10A illustrates the device 700 in a planar state, and FIG. 10B illustrates the device 700 in an expanded state.

The cross-sectional views of FIGS. 10A-10B enable viewing of the external threading 1024 of the threaded portion 804 of the rigid elongated member 702. The cross-sectional views further enable viewing of the corresponding internal threading 1030 disposed within the adjustment coupler 718. When the rigid elongated member 702 is in a fully planar state as shown in FIG. 10A, the external threading 1024 is located at a distal end of the internal threading 1030. When the rigid elongated member 702 is in a fully expanded state as shown in FIG. 10B, the external threading 1024 is located at a proximal end of the internal threading 1030. Additionally, in the fully expanded state illustrated in FIG. 10B, the shank 824 of the threaded portion 804 is disposed within the adjustment coupler 718 and cannot be seen from outside the device 700.

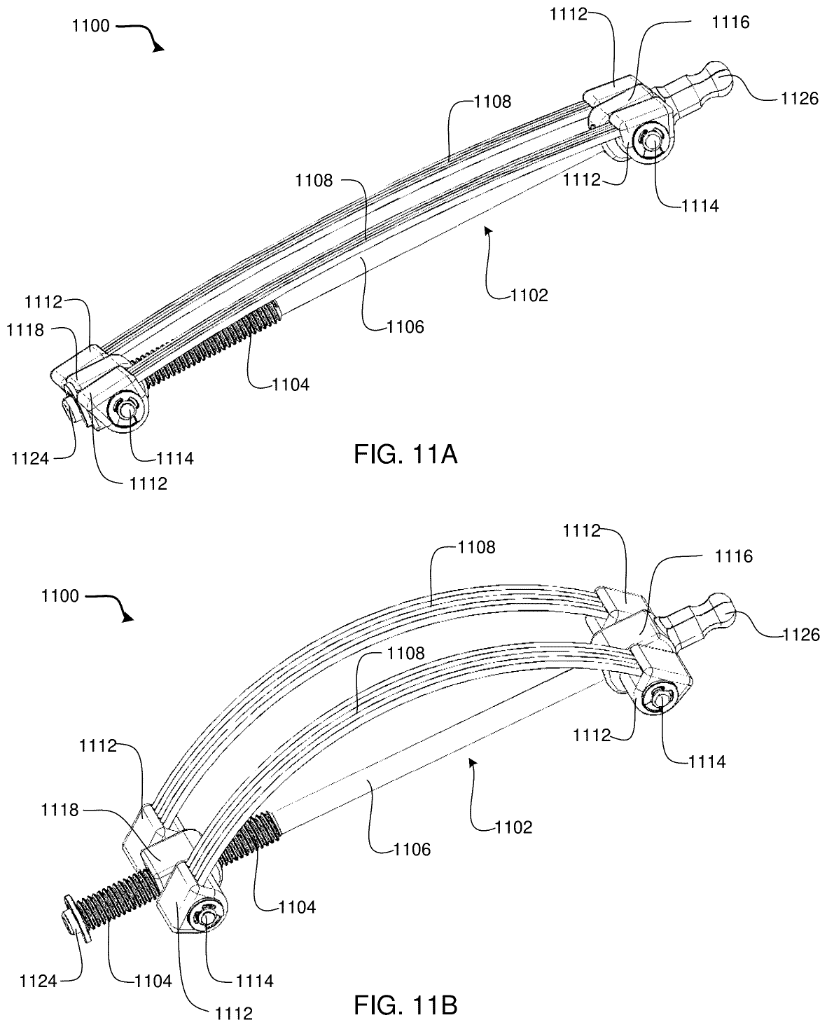

FIGS. 11A and 11B are overhead perspective views of a device 1100 for performing a distraction histogenesis procedure. FIG. 11A illustrates wherein the device 1100 is in a planar state, and FIG. 11B illustrates wherein the device 1100 is in an expanded state. The device 1100 may specifically be utilized to perform a periosteal distraction osteogenesis procedure to lift a periosteum tissue layer away from a bone tissue. The device 1100 may be utilized for other distraction histogenesis procedures, including those performed on soft tissues.

The device 1100 performs similar functions to the devices 100, 700 first described in connection with FIGS. 1A-1B and FIGS. 7A-7B, respectively. However, the device 1100 is not designed for magnetic coupling with an EMC. Instead, the device 1100 is designed to be distracted and retracted with a driver that directly interfaces with the device 1100. The device 1100 may be manually distracted and retracted by a user or the device 1100 may be automatically distracted and retracted by an automated adjustor with the same interface.

The device 1100 includes a rigid elongated member 1102. The rigid elongated member 1102 includes a threaded portion 1104 and may additionally include a smooth portion 1106. In some cases, the entirety of the rigid elongated member 1102 may include external threading like the threaded portion 1104.

The device 1100 includes a stationary coupler 1116 attached to the rigid elongated member 1102 at a first end of the rigid elongated member 1102. The stationary coupler 1116 is coupled to a pivot head 1112 by way of a pivot fastener 1114. The pivot fastener 1114 enables the pivot head 1112 to rotate about the center of the pivot fastener 1114. The pivot fastener 1114 may include, for example, a bearing bolt, axle screw, pivot bolt, rotating bolt, spindle bolt, turning screw, and so forth.

The device 1100 further includes an adjustment coupler 1118 attached to the rigid elongated member 1102 at a second end of the rigid elongated member 1102, which is opposite to the first end of the rigid elongated member 1102. Like the stationary coupler 1116, the adjustment coupler 1118 is also coupled to a pivot head 1112 by way of a pivot fastener 1114.

The device 1100 includes one or more compliant elongated members 1108 capable of bending away from a longitudinal axis of the rigid elongated member 1102. In the view illustrated in FIG. 11B, the compliant elongated members 1108 are bending away from the rigid elongated member 1102 to form an arch configuration or “distracted” configuration. The example device 1100 illustrated in FIGS. 11A and 11B includes a first compliant elongated member 1108 disposed on a first side of the rigid elongated member 1102, and a second compliant elongated member 1108 disposed on an opposite side of the rigid elongated member 1102. The compliant elongated members 1108 are coupled to the stationary coupler 1116 and the adjustment coupler 1118 by way of the pivot heads 1112 as shown in FIGS. 11A and 11B.

The adjustment coupler 1118 includes a hole defined by a sidewall, with internal threading attached to the sidewall (not visible in FIGS. 11A and 11B). The internal threading of the adjustment coupler 1118 corresponds with the external threading on the threaded portion 1104 of the rigid elongated member 1102, such that rotation of the rigid elongated member 1102 causes the adjustment coupler 1118 to move back and forth along the longitudinal axis of the rigid elongated member 1102.

The device 1100 includes a rotation actuation head 1124 attached to an end of the rigid elongated member 1102 that provides a means to rotate the rigid elongated member 1102 about its longitudinal axis (i.e., a “roll” rotation). The rotation actuation head 1124 may include an impression configured to receive a driver head. In these cases, the rotation actuation head 1124 may include, for example, an impression corresponding with a driver head type, such as a slot, coin-slot, hi-torque, cross, pozidriv, supadriv, Phillips, Frearson, French recess, torq-set, mortorq, Robertson, or other driver type. The rotation actuation head 1124 may include a socket head and may specifically include a polygonal cross-sectional geometry configured to interface with a socket. In these cases, the rotation actuation head 1124 may include, for example, a quadrilateral, pentagonal, hexagonal, heptagonal, octagonal, or n-sided cross-sectional geometry configured to interface with a corresponding socket.

The device 1100 is in a planar or retracted state when the distance between the stationary coupler 1116 and the adjustment coupler 1118 is at a maximum. The device 1100 is in a bent or expanded state when the distance between the stationary coupler 1116 and the adjustment coupler 1118 is less than the maximum. When this distance is less than the maximum, the compliant elongated members 1108 will bend away from the longitudinal axis of the rigid elongated member 1102 as shown in FIG. 11B.

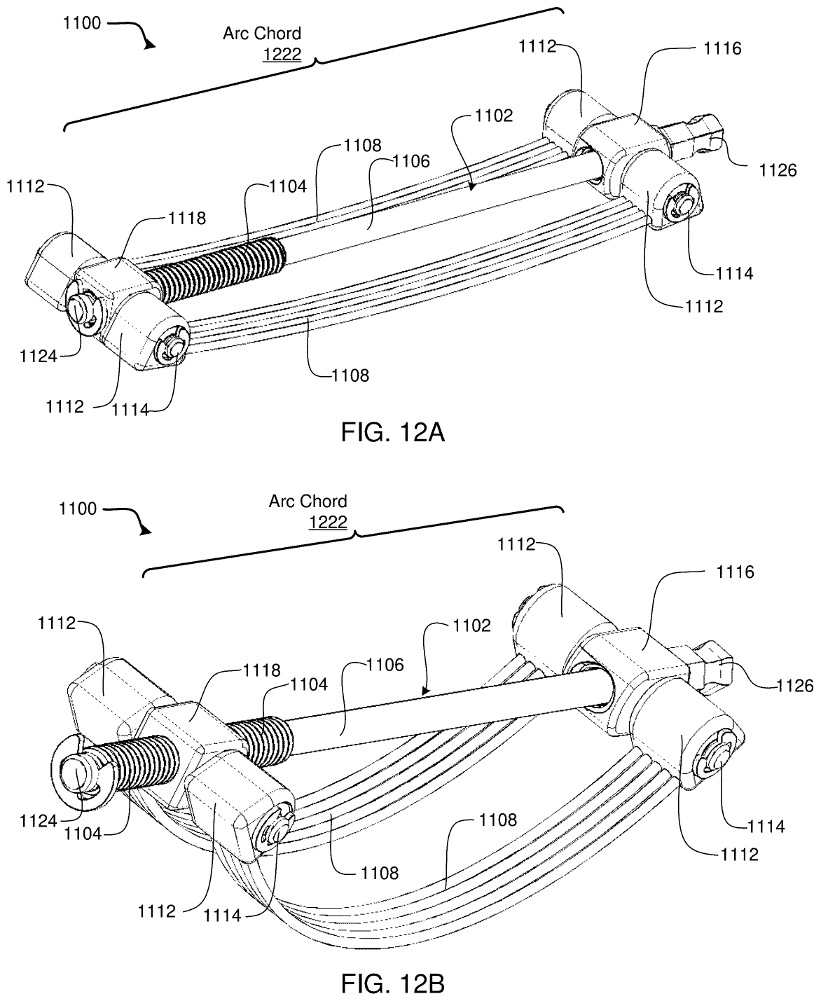

FIGS. 12A and 12B are underside perspective views of the device 1100 for performing a distraction histogenesis procedure, wherein FIG. 12A illustrates the device 1100 in a planar state, and FIG. 12B illustrates the device 1100 in an expanded state.

FIGS. 12A and 12B bring attention to the arc chord 1222, which is a distance between opposite end points of the compliant elongated member 1108. The arc chord 1222 may be measured from the pivot fastener 1114 of the adjustment coupler 1118 to the pivot fastener 1114 of the stationary coupler 1116 as shown in FIGS. 12A-12B. However, the arc chord 1222 may be measured from other endpoints as deemed appropriate. The arc chord 1222 refers to the straight point-to-point distance of the arc formed by the compliant elongated member 1108. When the device 1100 is in a planar state as shown in FIG. 12A, the arc chord 1222 comprises a longer length. When the device 1100 is in an expanded state as shown in FIG. 12B, the arc chord 1222 comprises a shorter length.

As shown in FIGS. 12A and 12B, the threaded portion 1104 of the rigid elongated member 1102 may screw into and out of the adjustment coupler 1118 to alter the position of the adjustment coupler 1118 along a length of the rigid elongated member 1102. When the threaded portion 1104 is minimally screwed into the adjustment coupler 1118 as shown in FIG. 12A, the arc chord 1222 comprises a maximum length. When the threaded portion 1104 is screwed further into the adjustment coupler 1118 as shown in FIG. 12B, the arc chord 1222 comprises a shorter length.

FIGS. 12A and 12B additionally provide a view of the rotation actuation head 1124, which enables a user to drive rotation of the rigid elongated member 1102. A user may manually drive rotation of the rigid elongated member 1102 with a driver or other device that is installed in the patient along with the device 100, or with a driver that is temporarily coupled to the rotation actuation head 1124. The user may actuate an automated fixator adjuster to autonomously drive rotation of the rigid elongated member 1102. The automated fixator adjuster may be installed in the patient along with the device 100, may be installed external to the patient with a coupling to the rotation actuation head 1124, or may be temporarily coupled to the rotation actuation head 1124 as needed. The automated fixator adjuster may include a driver and a motor to precisely rotate the rigid elongated member 1102 to thereby cause distraction or retraction of the compliant elongated member 1108.

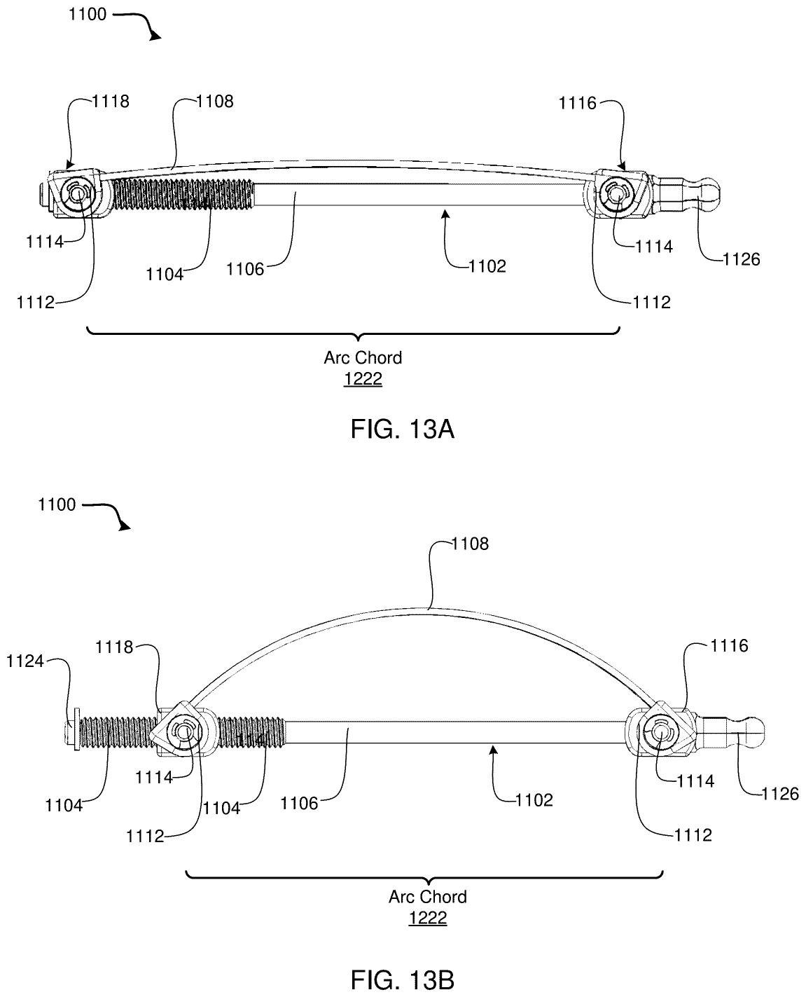

FIGS. 13A and 13B are straight-on side views of the device 1100 for performing a distraction histogenesis procedure, wherein FIG. 13A illustrates the device 1100 in a planar state, and FIG. 13B illustrates the device 1100 in an expanded state.

FIGS. 13A and 13B illustrate rotation of the pivot heads 1112 about the pivot fasteners 1114. When the total length of the arc chord 1222 is shortened by screwing the threaded portion 1104 through the adjustment coupler 1118 as shown in FIG. 13B, the compliant elongated member 1108 will form a taller arch away from the rigid elongated member 1102.

The length of the compliant elongated member 1108 may be optimized such that the compliant elongated member 1108 arches slightly away from the rigid elongated member 102 even when the arc chord 1222 is at its maximum length, as shown in FIG. 13A. Thus, the compliant elongated member 1108 may comprise a length that is slightly longer than the distance between the coupling points of the stationary coupler 116 and the adjustment coupler 118. This may be desirable to ensure the compliant elongated member 1108 arches in the desired direction when the device 1100 is distracted as shown in FIG. 13B.

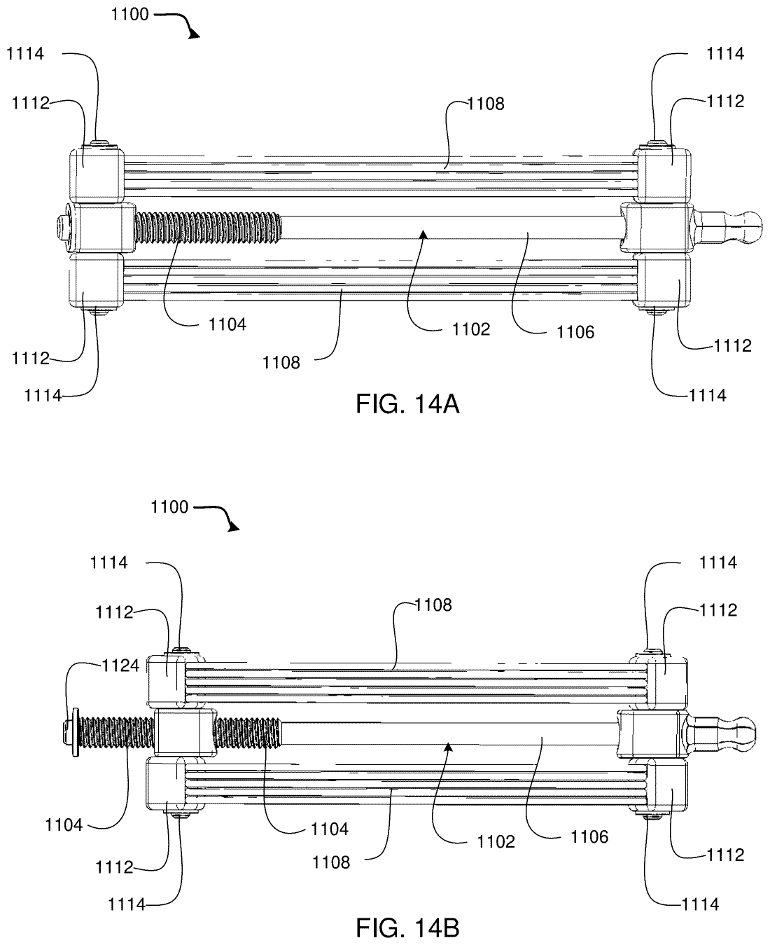

FIGS. 14A and 14B are top-down aerial views of the device 1100 for performing a distraction histogenesis procedure, wherein FIG. 14A illustrates the device 1100 in a planar state, and FIG. 14B illustrates the device 1100 in an expanded state.

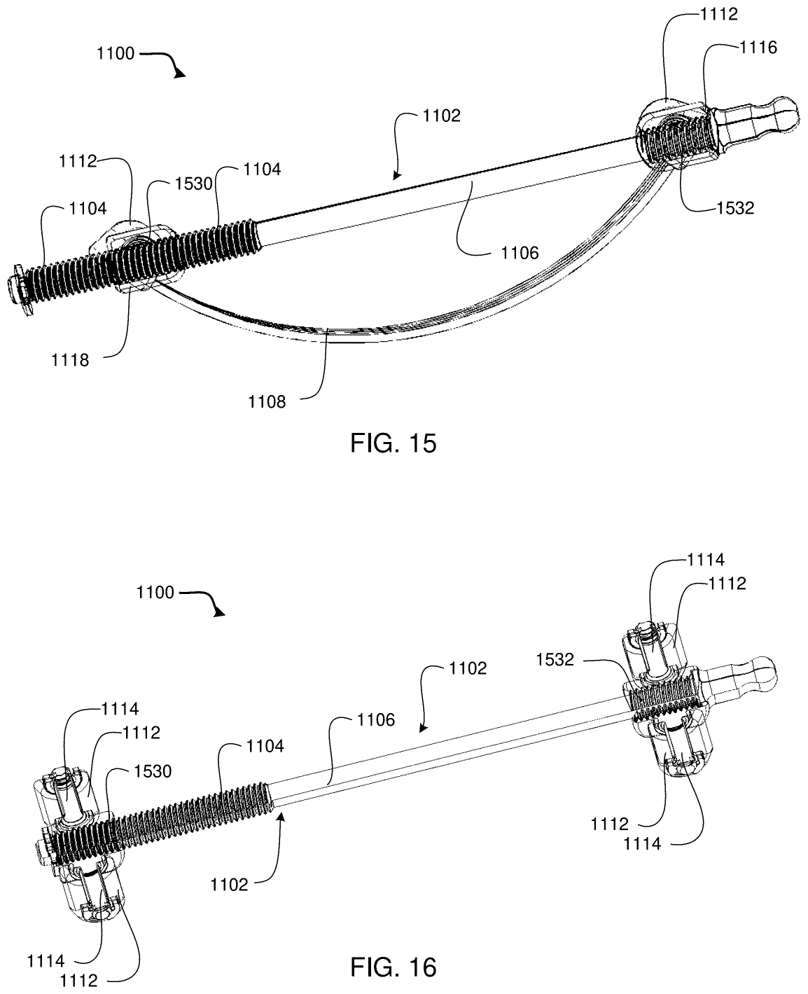

FIG. 15 is a perspective cross-sectional view of the device for performing a distraction histogenesis procedure. The cross-sectional slice is formed along an axis such that only one compliant elongated member 1108 is visible, and further to enable visualization into the couplers 1116, 1118.

As shown in the cross-sectional view of FIG. 15, the adjustment coupler 1118 includes a hollow interior defined by a sidewall and may include internal threading 1530 attached to the interior surface of the sidewall. The internal threading 1530 of the adjustment coupler 1118 corresponds with the external threading of the threaded portion 1104 of the rigid elongated member 1102. Thus, rotation of the rigid elongated member 1102 may cause the threaded portion 1104 to screw into or out of the sidewall of the adjustment coupler 1118.

The stationary coupler 1116 may further include a hollow interior defined by a sidewall that comprises internal threading 1532 attached to the interior surface of the sidewall. The rigid elongated member 1102 may include a secondary threaded portion that is configured to interface with the internal threading 1532 of the stationary coupler 1116. This interfacing may enable installation of the rigid elongated member 1102 within the stationary coupler 1116.

FIG. 16 is a perspective cross-sectional view of the device for performing a distraction histogenesis procedure. The cross-sectional slice is formed along the y-axis such that no compliant elongated members (see 1108) are visible, and further to enable visualization into the couplers 1116, 1118 and the pivot fasteners 1114.

As shown in the cross-sectional view of FIG. 16, the pivot fasteners 1114 may include rods extending a width of the pivot heads 1112. The pivot fasteners 1114 enable the pivot heads 1112 to rotate about the central point defined by the pivot fasteners 1114. The pivot motion of the pivot heads 1112 may enable corresponding pivoting by the compliant elongated member, which enables the compliant elongated member to distract away from the rigid elongated member 1102 without forming a kink or sharp bend.

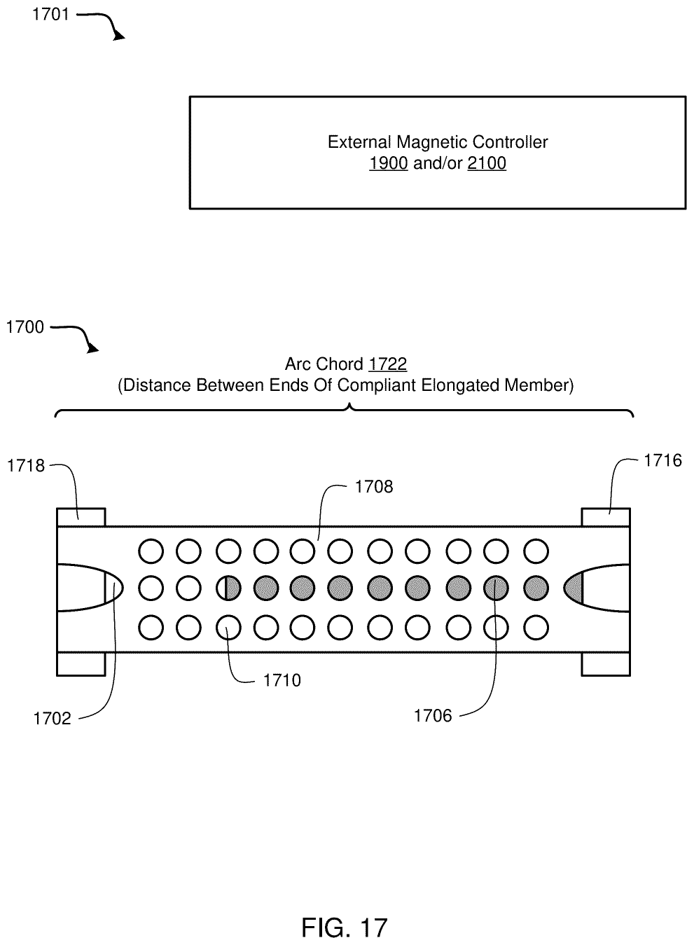

FIG. 17 is a schematic illustration of a system 1701 for performing a distraction surgical procedure, and specifically illustrates a straight-on aerial top-down view of a device 1700 for performing the distraction surgical procedure, wherein the device 1700 is a component of the wholistic system 1701. The device 1700 is configured to be actuated through a magnetic coupling. The device 1700 may include any of the components described in connection with the device 100 first described in connection with FIGS. 1A and 1B. The device 1700 may further include any of the components described in connection with the device 700 first described in connection with FIGS. 7A and 7B.

The device 1700 includes a rigid elongated member 1702 that includes a magnetic portion 1706 (see, e.g., the magnetic portion 106 first described in connection with FIGS. 1A-1B or the magnetic portion 706 first described in connection with FIGS. 7A-7B). The device 1700 includes a compliant elongated member 1708 that is coupled to the rigid elongated member 1702 by way of two or more couplers 1716, 1718. The compliant elongated member 1708 is configured to bend away from a longitudinal axis of the rigid elongated member 1702. In the view illustrated in FIG. 17, the compliant elongated member 1708 would bend out of the page away from the rigid elongated member 1702, which is disposed underneath the compliant elongated member 1708. The arc chord 1722 corresponds with the distance between opposing ends of the compliant elongated member 1708.

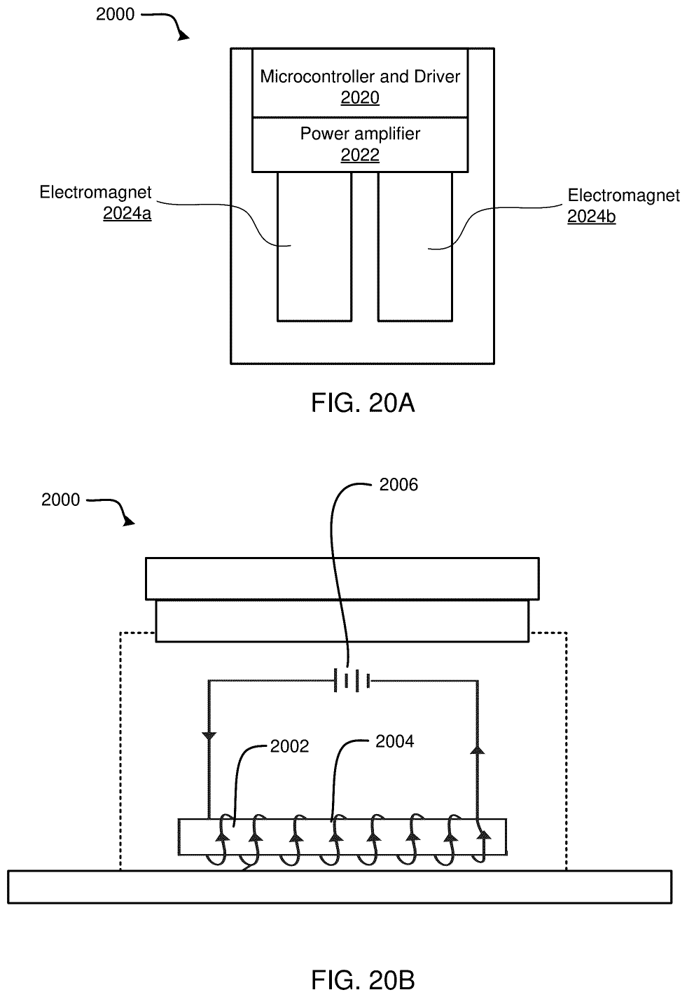

The system 1701 is designed such that the device 1700 may be installed within a patient body, and the patient's body may be closed to prevent infection (e.g., the patient's skin may be sutured or otherwise resealed after installing the device 1700). The system 1701 includes an EMC (see, e.g., EMC 1900 illustrated at least at FIGS. 19A-19C, or EMC 2000 illustrated at least at FIGS. 20A-20B) that is configured to be located external to the patient's body such that components within the EMC 1900, 2000 wirelessly interface with the magnetic portion 1706 of the rigid elongated member 1702 through patient tissue layers, such as skin, fascia, fat, periosteum, and so forth.

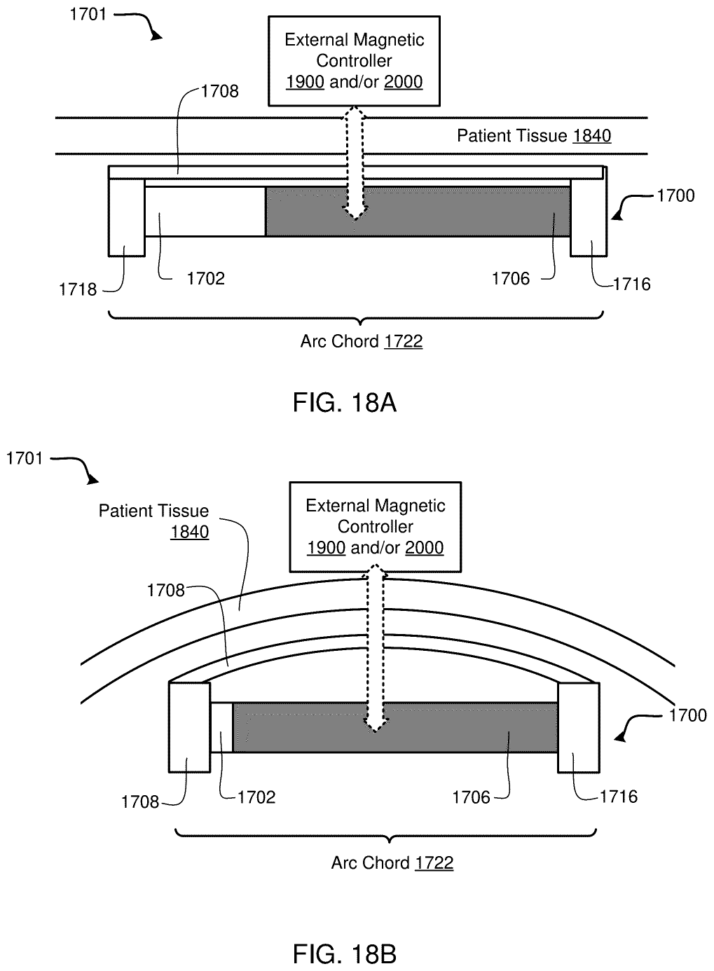

FIGS. 18A and 18B are schematic illustrations of straight-on side views of the system 1701 and device 1700 for performing a distraction surgical procedure. FIG. 18A illustrates wherein the compliant elongated member 1708 of the device 1700 is in a relaxed position such that the arc chord 1722 is at the maximum length. FIG. 18B illustrates wherein the compliant elongated member 1708 of the device 1700 is in a bent state such that the arch length 1722 is less than the maximum.

As shown in FIGS. 18A-18B, the device 1700 may be installed underneath patient tissue 1840, which may include, for example, periosteum, fascia, fat, muscle, skin, and other tissues. The patient tissue 1840 may be closed after installation of the device 1700 to prevent infection. The EMC 1900, 2000 may then be located externally to the patient body and placed near the device 1700 to establish a magnetic coupling between the EMC 1900, 2000 and the device 1700 that is installed underneath the patient tissue 1840.

The magnetic force of the EMC 1900, 2000 is sufficiently strong to cause rotation of the magnetic portion 1706 of the rigid elongated member 1702. The rotation of the rigid elongated member 1702 thereby causes an adjustment in the length of the arc chord 1722 and adjustment to the height of the arc formed by the compliant elongated member 1708. This is described in connection with the devices 100, 700 first described in connection with FIGS. 1A-1B and FIGS. 7A-7B, respectively.

The EMC 1900, 2000 need not necessarily be placed over the device 1700 as shown in FIGS. 18A-18B. The EMC 1900, 2000 may be placed alongside the device, over top the device, or otherwise near the device to establish a magnetic coupling between the EMC 1900, 2000 and the device 1700. If the magnet of the EMC 1900, 2000 is substantially parallel to an elongated magnet of the device 1700, the magnetic coupling may be established successfully. In some cases, the EMC 1900, 2000 may include multiple magnets, and one or more of those multiple magnets may form a substantially parallel magnetic coupling with the elongated magnet of the device 1700.

FIGS. 19A-19C are schematic illustrations of an EMC 1900 that includes an external magnet. FIG. 19A is a schematic illustration of an aerial top-down view of an EMC 1900 that may be mechanically adjusted by machine or user. FIG. 19B is a schematic illustration of a cross-sectional straight-on side view of an EMC 1900 that may be mechanically adjusted by a machine or user. FIG. 19C is a schematic illustration of a cross-sectional straight-on side view of an EMC 1900 that may be electronically adjusted with a microcontroller, driver, and motor.

The EMCs described herein may include one or more of a magnet or an electromagnet, and in some cases, an EMC may include each of a magnet and an electromagnet. Any of the EMCs described herein may include any quantity of magnets or electromagnets as deemed appropriate for the particular use-case. Any of the EMCs described herein, including any of those illustrated in FIGS. 19A-19C, 20A-20B, 21A-21F, and 22A-22B, may be utilized in connection with any of magnetically actuated arc lifter distraction devices described herein, including the device 100 first illustrated in connection with FIGS. 1A-1B and the device 700 first illustrated in connection with FIGS. 7A-7B. The arc lifter distraction devices described herein may be coupled to an EMC with an external magnet (see, e.g., EMC 1900) and/or an EMC with an electromagnet (see, e.g., EMC 2000).

FIG. 19A is a schematic illustration of an aerial top-down view of the EMC 1900. The EMC 1900 includes a magnet or electromagnet that forms a magnetic coupling with the magnetic portion (see, e.g., 106, 706, 1706) of the distraction device (see, e.g., 100, 700, 1700). The EMC 1900 may be utilized to cause rotation of a rigid elongated member (see, e.g., 102, 702, 1702) without physically touching the rigid elongated member. The EMC 1900 may thus be utilized to adjust a length of an arc chord and thereby adjust a height of an arc configuration formed by a compliant elongated member (see, e.g., 108, 708, 1708). Thus, a patient or healthcare provider may wirelessly distract and retract an arc lifter distraction device installed in a patient when the patient's skin barrier is sutured shut.

The EMC 1900 may include a housing that comprises a dial 1902 that may be rotated clockwise and counterclockwise. In the example illustrated in FIG. 19A, the dial 1902 may be rotated clockwise to rotate in a positive 1904 direction, which would further distract a compliant elongated member. The dial 1902 may be rotated counterclockwise in a negative 1906 direction, which would retract the compliant elongated member.

FIG. 19B is a schematic illustration of a straight-on cross-sectional view of the EMC 1900. The EMC 1900 includes an external magnet 1910 that includes at least a first polarity 1912 and a second polarity 1914. The external magnet 1910 is described as “external” because it may be located external to the patient's body when the EMC 1900 is utilized to distract or retract a magnetically actuation arc lifter distraction device. Like the magnetic portion (see, e.g., 106, 706, 1706) of the magnetically actuated arc lifter distraction devices described herein, the external magnet 1910 is arranged such that each of the first polarity 1912 and the second polarity 1914 are disposed along a length of the elongated magnet. The external magnet 1910 may include a cylindrical geometry as shown in FIG. 19B, and in this implementation, the first polarity 1912 and the second polarity 1914 run the entire length of the elongated cylindrical geometry as shown in FIG. 19B.

The EMC 1900 may include more than two poles. In some cases, it may be desirable to include an elongated magnet that comprises more than two poles to provide a finer ability to cause corresponding rotation of the magnetic portion of a rigid elongated member. The systems described herein may be arranged such that elongated magnet of the EMC 1900 includes the same quantity of poles as the magnetic portion of a rigid elongated member.

FIG. 19C is a schematic illustration of a cross-sectional straight-on side view of an EMC 1900 that includes a microcontroller for driving rotation of an external magnet 1910. The EMC 1900 includes the external magnet 1910 having the first polarity 1912 and the second polarity 1914. The EMC 1900 additionally includes a microcontroller and driver 1980 in electronic communication with a motor 1920. The motor 1920 is in mechanical communication with a driver interface 1922, and the driver interface 1922 is in mechanical communication with a corresponding socket interface 1924 attached to the external magnet 1910. The microcontroller and driver 1980 instructs the motor 1920 to cause rotation of the driver interface 1922, and this may in turn cause rotation of the socket interface 1924 and the external magnet 1910.

FIGS. 20A-20B are schematic illustrations of an EMC 2000 that includes one or more electromagnets for driving rotation of an implant magnet. FIG. 20A is a schematic illustration of a straight-on cross-sectional view of an EMC 2000 that includes one or more electromagnets 2024a, 2024b. The EMC 2000 may include a microcontroller and driver 2020 in electronic communication with the electromagnets 2024a, 2024b. The EMC 2000 may additionally include a power amplifier 2022 in electronic communication with the electromagnet 2024a, 2024b and the microcontroller and driver 2020, wherein the power amplifier 2022 powers a coil of the electromagnets 2024a, 2024b.

FIG. 20B is a schematic illustration of a straight-on cross-sectional view of an EMC 2000 that includes an electromagnet in lieu of an external magnet. The EMC 2000 includes an electromagnet comprising a rod 2002, a coil 2004 wrapped around the rod 2002, and a battery 2006. The coil 2004 may be driven by a power amplifier and controller by a microcontroller.

FIGS. 21A-21F are schematic illustrations of a system 2100 for driving an implant magnet 2106 of an arc lifter distraction device with an EMC 2000 that includes electromagnets. FIGS. 21A-21F include a schematic illustration of a cross-sectional straight-on side view of the EMC 2000, and additionally include an aerial top-down view of the implant magnet 2106 of the arc lifter distraction device to provide context to the current rotational position of the implant magnet 2106.

The system 2100 includes the implant magnet 2106, which is a component of an arc lifter distraction device and is implanted in a patient. The implant magnet 2106 may include a magnet of the magnetic portion 106 of the rigid elongated member 102 first described in connection with FIGS. 1A-1B. The implant magnet 2106 may include a magnet of the magnetic portion 706 of the rigid elongated member 702 first described in connection with FIGS. 7A-7B. The implant magnet 2106 includes at least a first pole 2108 and a second pole 2110. The implant magnet 2106 may include more than two poles, and may specifically include four, six, eight, or more poles.

The system 2100 is arranged such that the EMC 2000 includes a two-pole electromagnet 2102 comprising a first coil 2120 and a second coil 2122. The EMC 2000 includes a Hall effect sensor or Hall effect array 2124 configured to track a current orientation of the implant magnet 2106 of the distraction device. The system 2100 may be implemented with a two pole electromagnet 2102 that is connected as shown in FIGS. 21A-21F. The system 2100 may alternatively be implemented with a two pole electromagnet 2102 that comprises two separate (non-connected) electromagnets.

The schematic illustrations of FIGS. 21A-21F describe a principle of traveling waves that enable granular rotation of the implant magnet 2106 by engaging the two-pole electromagnet 2102 of the EMC 2000. The two-pole electromagnet 2102 may be arranged as an array around the implant magnet 2106 when the EMC 2000 is placed on the patient's skin to form a magnetic coupling with the implant magnet 2106.

FIG. 21A illustrates an initiation orientation for the system 2100. The two-pole electromagnet 2102 includes the first coil 2120 and the second coil 2122. The EMC 2000 further includes the Hall effect sensor or Hall effect array 2124 to track the current rotational orientation of the implant magnet 2106. The first coil 2120 and the second coil 2122 are located at approximately the junction between the first pole 2108 and the second pole 2110 of the implant magnet 2106.

FIG. 21B illustrates a zero degree rotation orientation for the system 2100, wherein all components of the system are in the same configuration as described in connection with the initiation orientation of FIG. 21A.

FIG. 21C illustrates a 90 degree rotation orientation for the system 2100. The implant magnet 2106 has rotated 90 degrees relative to the initiation orientation illustrated in FIG. 21A. The second coil 2122 is engaged.

FIG. 21D illustrates a 180 degree rotation orientation for the system 2100. The implant magnet 2106 has rotated 180 degrees relative to the initiation orientation illustrated in FIG. 21A. The second coil 2122 is engaged.

FIG. 21E illustrates a 270 degree rotation orientation for the system 2100. The implant magnet 2106 has rotated 270 degrees relative to the initiation orientation illustrated in FIG. 21A. The second coil 2122 is engaged.

FIG. 21F illustrates a 360 degree rotation orientation for the system 2100. The implant magnet 2106 has the same rotational position as the initiation orientation illustrated in FIG. 21A. The first coil 2120 and the second coil 2122 are engaged.

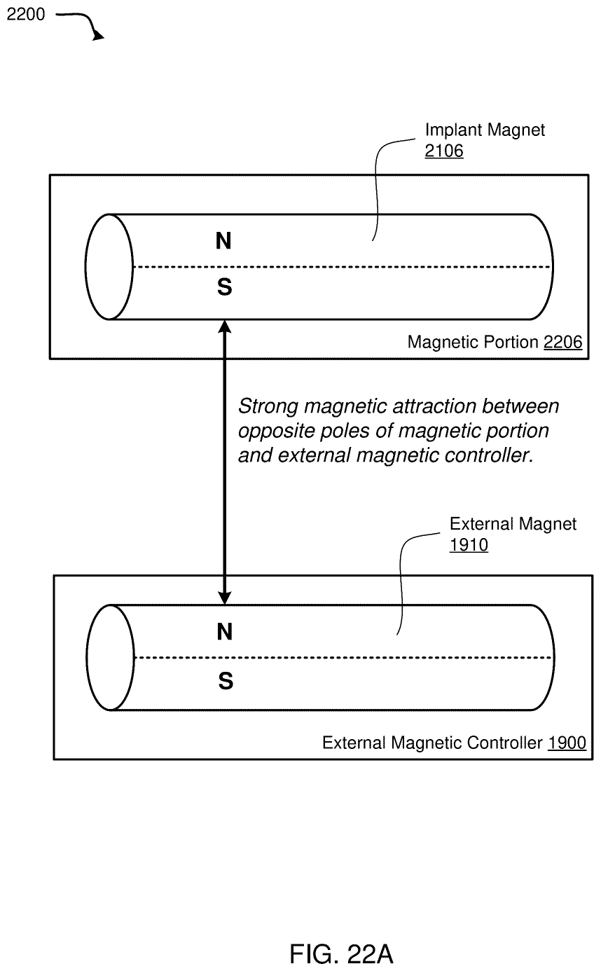

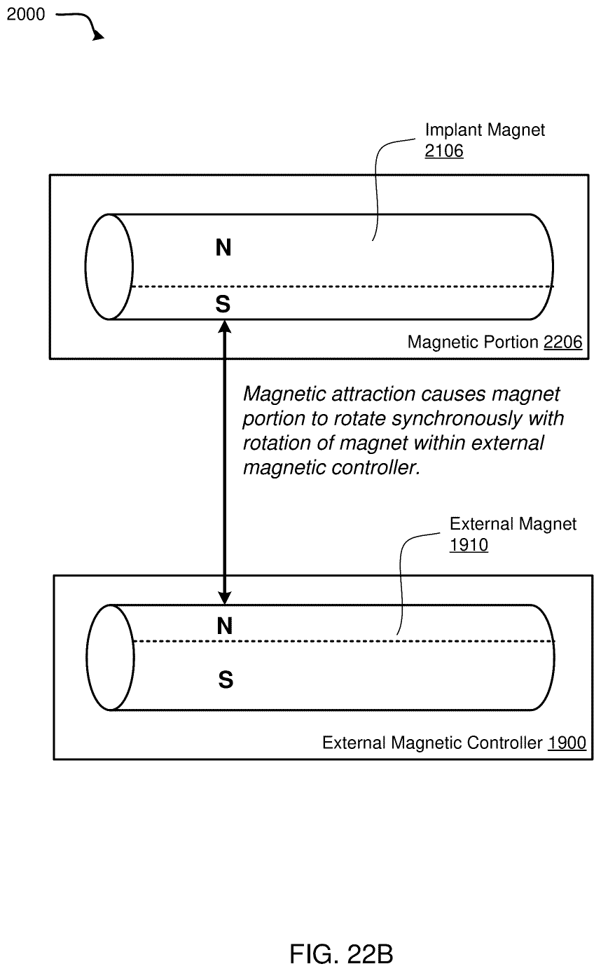

FIGS. 22A and 22B are schematic illustrations depicting the magnetic coupling between an implant magnet 2106 of a magnetic portion 2206 of an arc lifter distraction device, and an external magnet 1910 of an EMC 1900. The implant magnet 2106 may be implemented in any of the magnetic-actuation distraction devices described herein, including at least the device 100 first described in connection with FIGS. 1A-1B or the device 700 first described in connection with FIGS. 7A-7B.

As shown in FIG. 22A, a first polarity of the implant magnet 2106 establishes a magnetic coupling with a corresponding polarity of the external magnet 1910. In the example illustrated in FIGS. 22A-22B, the first polarity of the implant magnet 2106 is a South polarity, and the corresponding polarity of the external magnet 1910 is a North polarity.

As shown in FIG. 22B, rotation of magnet 1910 of the EMC 1900 causes synchronous rotation of the implant magnet 2106 of the magnetic portion 2206 due to the magnetic attraction of the corresponding diametrically magnetized magnetic poles. Thus, rotation of the external magnet 1910 of the EMC 1900 may cause rotation of a rigid elongated member of an arc lifter distraction device, which may thereby cause further arching of a compliant elongated member. A patient or healthcare provider may thus place the EMC 1900 externally on the patient over the implanted device, establish the magnetic coupling between the EMC 1900 and the magnetic portion 2206, and then distract or retract the arch of the compliant elongated member by rotating the external magnet 1910 of the EMC 1900.

Each of the implant magnet 2106 and the external magnet 1910 may include more than two poles. Typically, the implant magnet 2106 and the external magnet 1910 will include the same quantity of poles. In some cases, it may be desirable to ensure each of the implant magnet 2106 and the external magnet 1910 include more than two poles, such as four, six, eight, ten, or more poles. A greater quantity of poles may allow for more granular rotation of the implant magnet 2106 of the magnetic portion 2206, which may in turn allow for more granular rotation of a rigid elongated member.

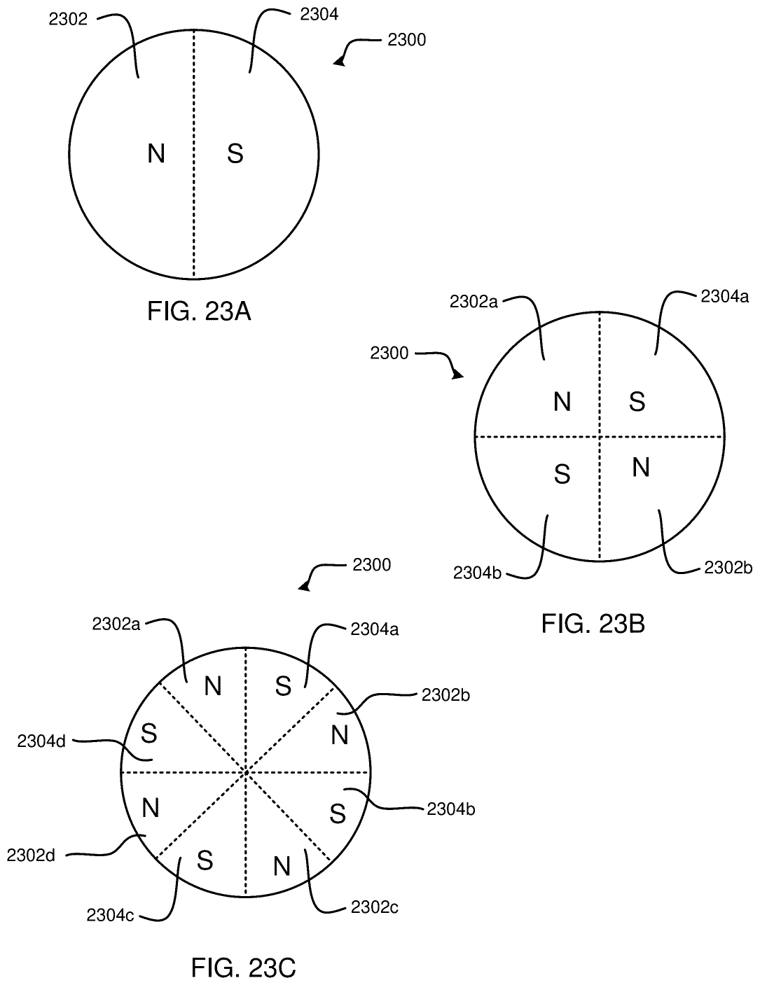

FIGS. 23A-23C are schematic cross-sectional aerial top-down views of exemplary magnets 2300 that may be utilized in connection with the devices described herein. The magnets 2300 may be implemented as the implant magnet of the magnetic portion (see 106, 706, 1706) of a rigid elongated member (see 102, 702, 1702) of a magnetically actuated arc lifter distraction device as described herein. The magnets 2300 may be implemented as the external magnet 1910 of an EMC 1900. FIGS. 23A-23C each illustrate an aerial top-down view of a top of a magnet 2300 or cross-sectional slice of the magnet 2300.

FIG. 23A illustrates an example wherein the magnet 2300 includes two poles, including a first pole 2302 including a first polarity and a second pole 2304 including a second polarity. The first polarity is opposite to the second polarity. The poles 2302, 2304 are adjacent to one another in the cross-sectional slice such that each of the poles 2302, 2304 comprises a half-circle cross-sectional geometry.

FIG. 23B illustrates an example wherein the magnet 2300 includes four poles, including two of a first pole 2302a, 2302b each having a first polarity. The magnet 2300 further includes two of a second pole 2304a, 2304b each having a second polarity that is opposite to the first polarity. The poles are arranged in an alternating fashion such that any of the first pole 2302a, 2302b is adjacent to two of the second pole 2304a, 2304b. The poles are further arranged such that each of the poles 2302a, 2302b, 2304a, 2304b comprises a circular sector (pie slice) cross-sectional geometry.

FIG. 23C illustrates an example wherein the magnet 2300 includes eight poles, including four of a first pole 2302a, 2302b, 2302c, 2302d each having a first polarity. The magnet 2300 further includes four of a second pole 2304a, 2304b, 2304c, 2304d each having a second polarity that is opposite to the first polarity. The poles are arranged in an alternating fashion such that any of the first pole 2302a, 2302b, 2302c, 2302d is adjacent to two of the second pole. Likewise, any of the second pole 2304a, 2304b, 2304c, 2304d is adjacent to two of the first pole. The poles are further arranged such that each of the poles 2302a-2302d, 2304a-2304d comprises a circular sector (pie slice) cross-sectional geometry.

The magnetically actuated arc lifter distraction devices (see, e.g., 100, 700, 1700) described herein may be implemented with a magnet that comprises a plurality of a first pole having a first polarity, and a plurality of second pole having a second polarity that is opposite to the first polarity. Additionally, the EMC 1900 described herein may include an external magnet 1910 that comprises a plurality of a first pole having a first polarity, and a plurality of second pole having a second polarity that is opposite to the first polarity. The systems described herein may be implemented such that the magnet of the distraction device includes the same quantity of poles as the external magnet of the EMC. In some cases, it may be desirable to include a greater quantity of poles to enable increased granularity when rotating the external magnet of the EMC, which causes synchronous rotation of the magnet of the distraction device, and which further causes synchronous rotation of the actuation screw of the distraction device.

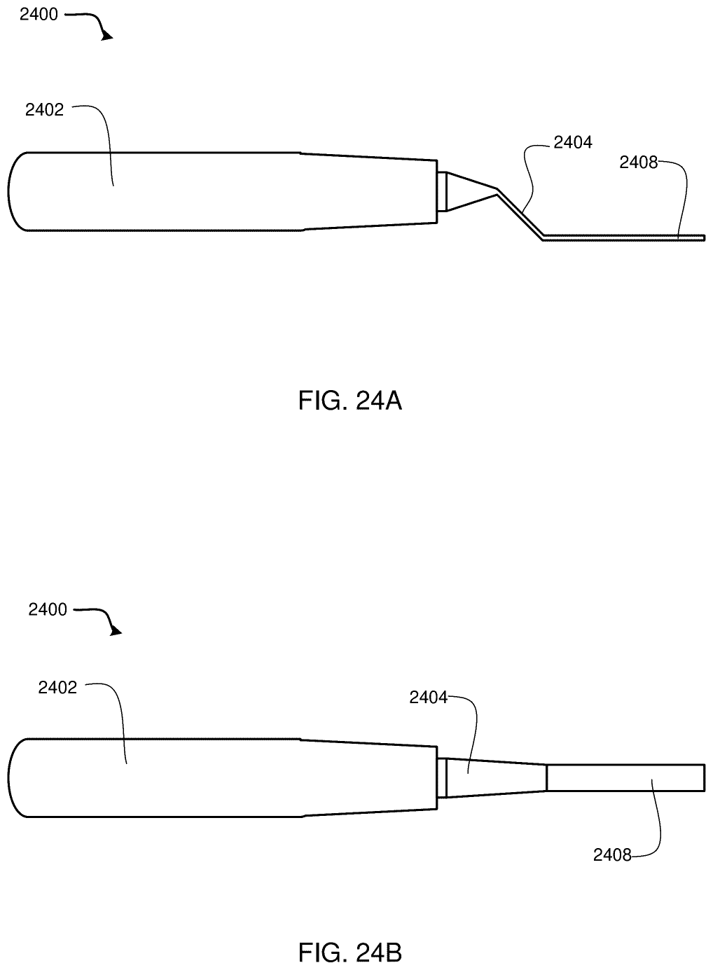

FIGS. 24A and 24B are schematic illustrations of an elevator 2400 for use in connection with performing a distraction surgical procedure. FIG. 24A illustrates a straight-on wide view of the elevator 2400, and FIG. 24B illustrates a straight-on top-down aerial view of the elevator 2400. The elevator 2400 may specifically be utilized as a periosteal elevator to lift a periosteal tissue away from an outer bone surface. In this case, the elevator 2400 may be utilized to perform a periosteal distraction osteogenesis procedure. The elevator 2400 may be utilized to perform other distraction procedures, including procedures performed on soft tissues.

The elevator 2400 includes a handle 2402, a neck 2404, and a blade 2408. Each of the neck 2404 and the blade 2408 may comprise a smooth and blunt edge to prevent the elevator 2400 from cutting patient tissue or causing additional tissue trauma. During a surgical procedure, a cut may be made through patient tissue that is approximately the length of the width of the blade 2408. The blade 2408 may then be inserted through the cut and utilized to lift patient tissue away.

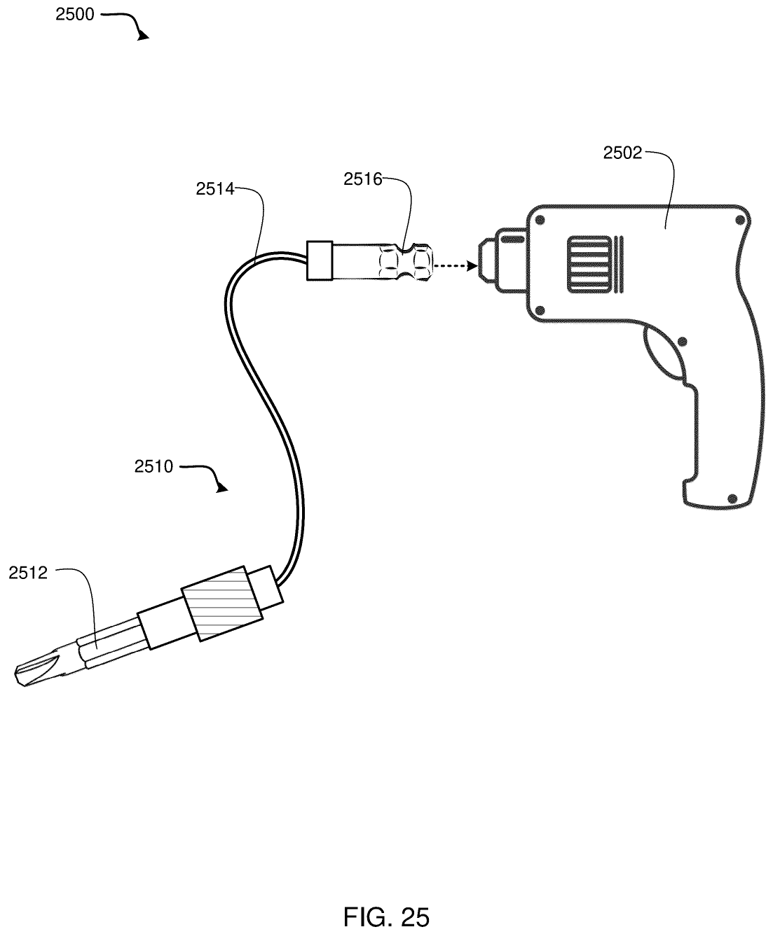

FIG. 25 is a schematic illustration of a system 2500 that includes a flexible driver 2510 for performing a distraction surgical procedure. The flexible driver 2510 includes a drill bit 2512, a flexible shank 2514, and a rigid shank 2516. The rigid shank 2516 is configured to be inserted into a corresponding receptacle of a drill 2502. The flexible shank 2514 enables a user to manipulate the position of the drill bit 2512 to more easily access a rotation actuation head (see, e.g., 1124) and rotate a rigid elongated member (see, e.g., 1102).

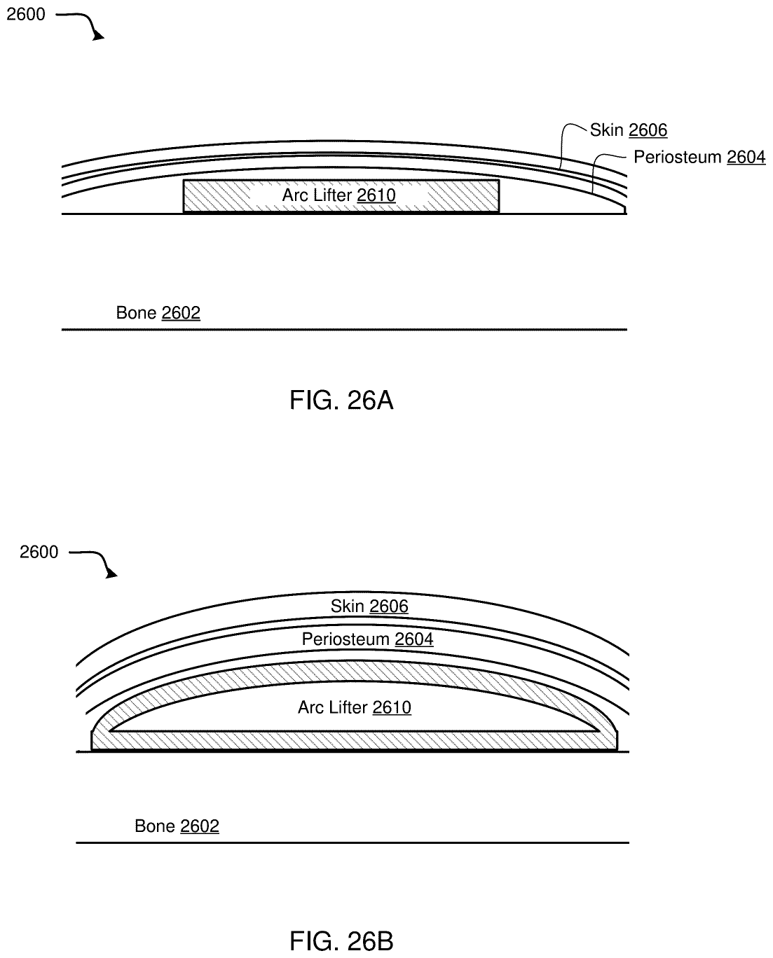

FIGS. 26A and 26B are schematic illustrations of a system 2600 for distraction histogenesis, and specifically illustrates wherein an arc lifter distraction device is installed in a patient. The system 2600 includes an arc lifter 2610 installed in between a patient bone 2602 and patient periosteum 2604, and further underneath at least skin 2606 of the patient. The arc lifter 2610 may include any of the arc lifter distraction devices described herein, including, for example, the device 100 first described in connection with FIGS. 1A-1B, or the device 700 first described in connection with FIGS. 7A-7B, or the device 1100 first described in connection with FIGS. 11A-11B 1.

FIG. 26A illustrates wherein the arc lifter 2610 is in a relaxed state, such that an arc chord is at a maximum. FIG. 26B illustrates wherein the arc lifter 2610 is in a bent state, such that the arc chord is less than the maximum. As shown in FIG. 26B, the bending of the arc lifter 2610 causes further distraction of the periosteum 2604 away from the bone 2602.

FIG. 26B illustrates wherein the patient skin 2606 is resealed, but a cut remains in the periosteum 2604 of the patient (see left side of FIG. 26B, wherein periosteum 2604 is not fully continuous). This system 2600 might be implemented when the arc lifter 2610 includes a magnet-actuated arc lifter configured to interface with an external magnetic controller (see, e.g., EMC 1900, 2000). In other cases, the skin 2606 may remain opened to allow a user to access the arc lifter 2610 with a flexible driver or other device.

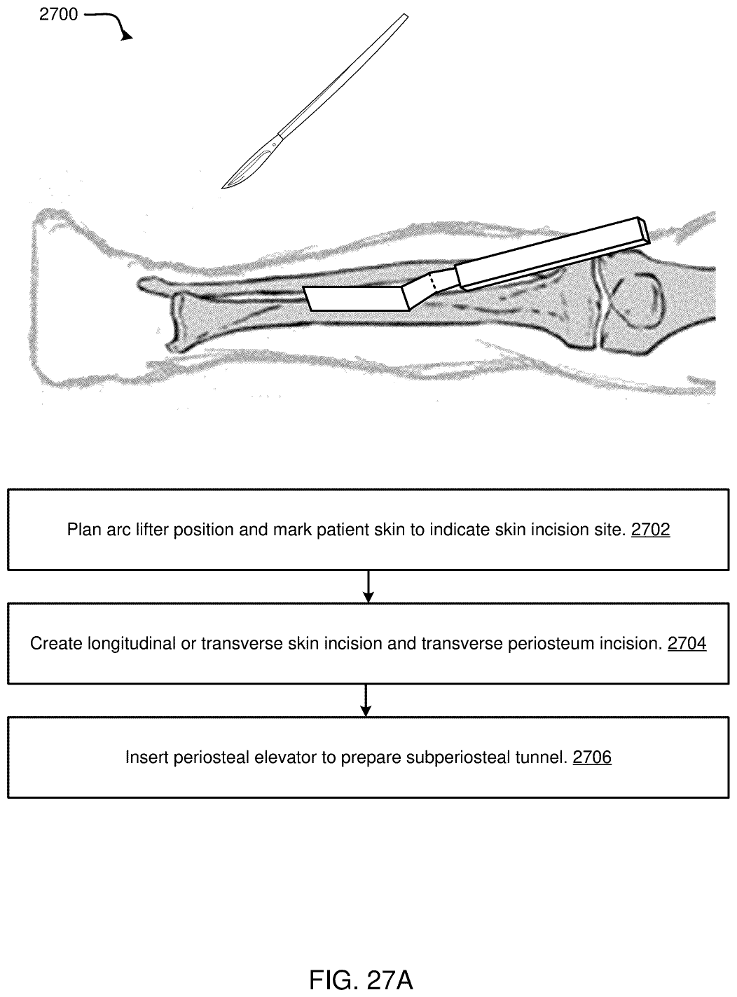

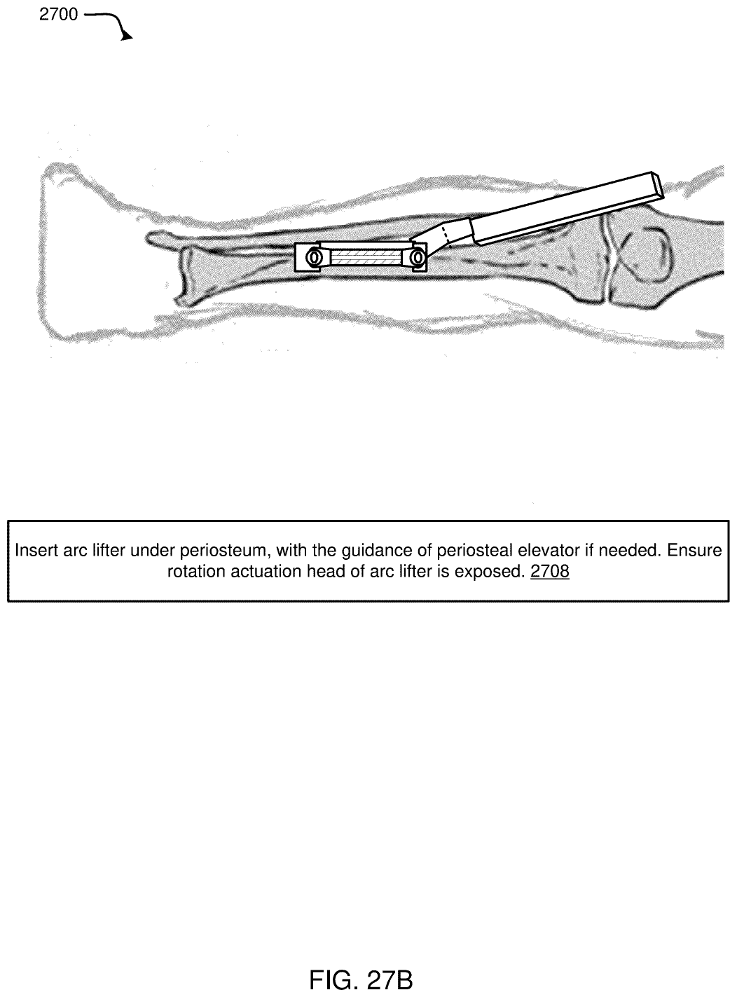

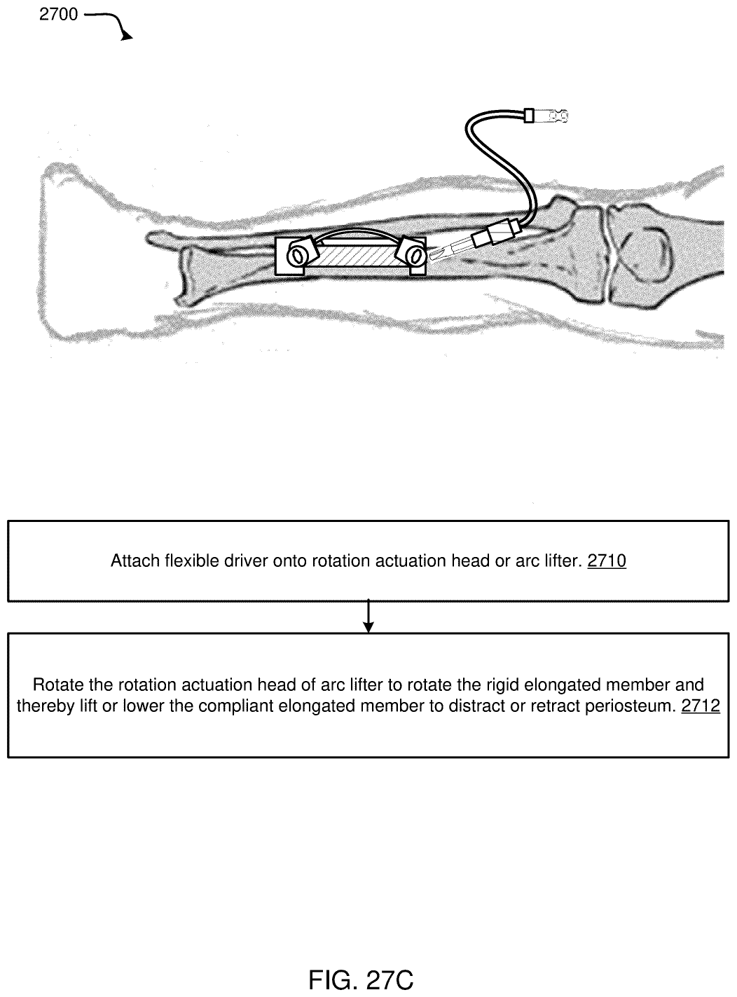

FIGS. 27A-27C are schematic block diagrams of a method 2700 for performing a periosteal distraction osteogenesis procedure. The method 2700 steps may be slightly modified to perform a similar distraction histogenesis procedure on alternative tissue types, including soft tissues. The method 2700 may be performed with an arc lifter distraction device as described herein, including at least the device 1100 first described in connection with FIGS. 11A-11B.

The method 2700 includes planning at 2702 an arc lifter position and marking the patient skin to indicate skin incision site. The method 2700 includes creating at 2704 a longitudinal or transverse skin incision and transverse periosteum incision. The method 2700 include inserting at 2706 a periosteal elevator to prepare subperiosteal tunnel. The method 2700 include inserting at 2708 an arc lifter under the patient periosteum, with the guidance of the periosteal elevator if needed. The method 2700 may be performed to ensure the rotation actuation head of the arc lifter is exposed. The method 2700 includes attaching at 2710 a flexible driver (or flexible socket, or other driver or socket, or other means for actuating rotation actuation head) onto the rotation actuation head of the arc lifter. The method 2700 includes rotating at 2712 the rotation actuation head of the arc lifter to thereby rotate the rigid elongated member of the arc lifter, and to thereby lift or lower the compliant elongated member, and thereby distract or retract the patient periosteum.

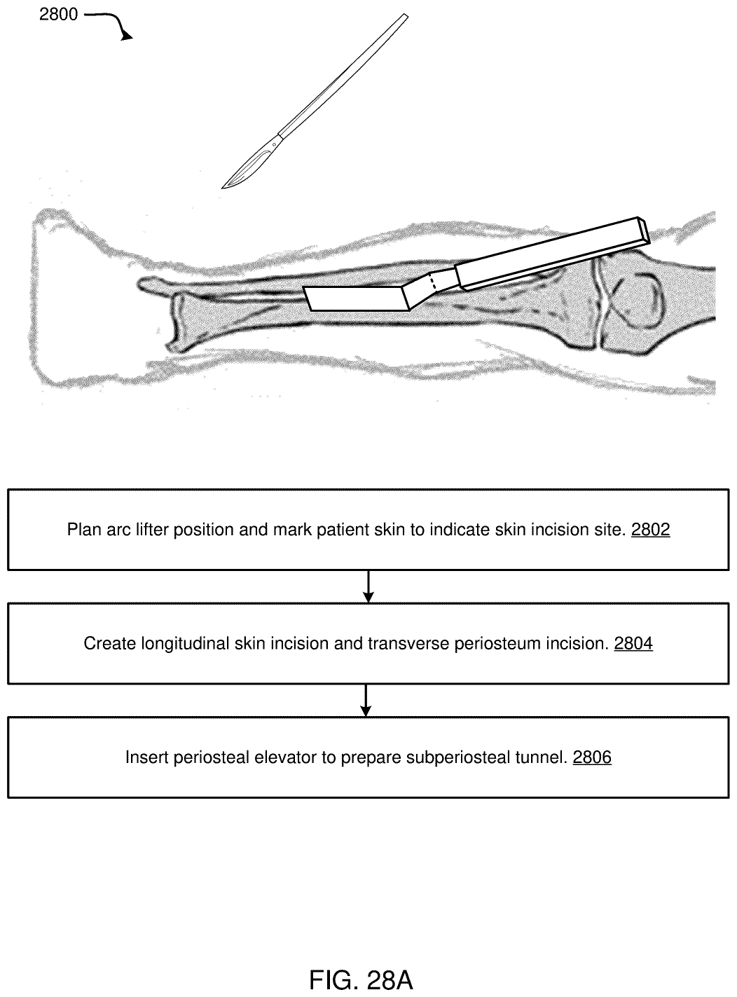

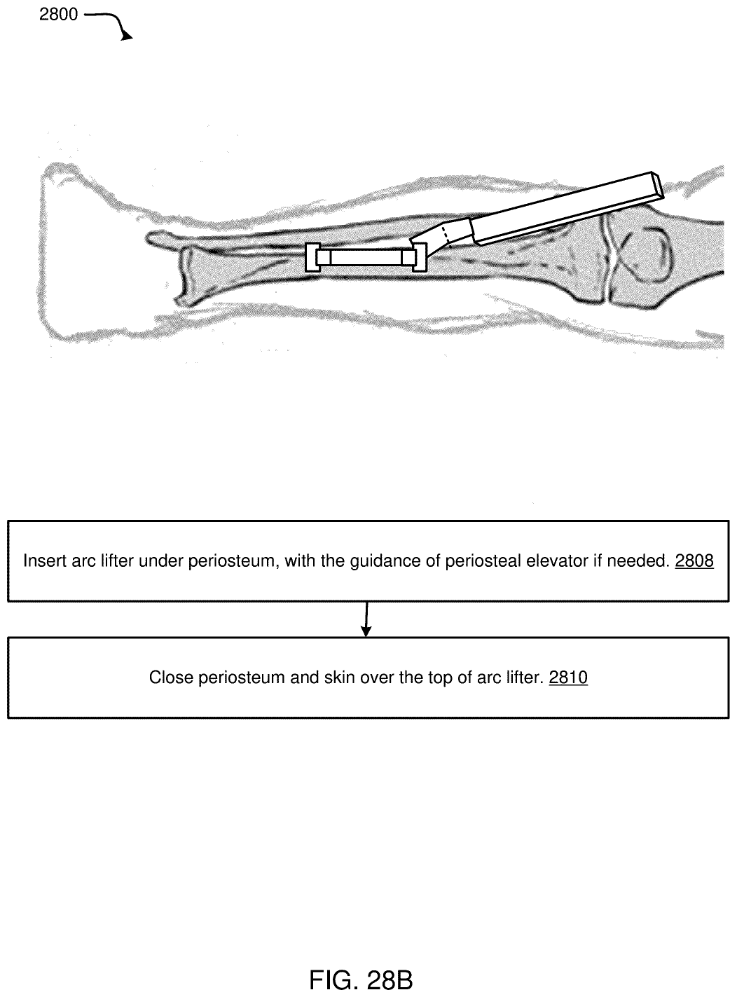

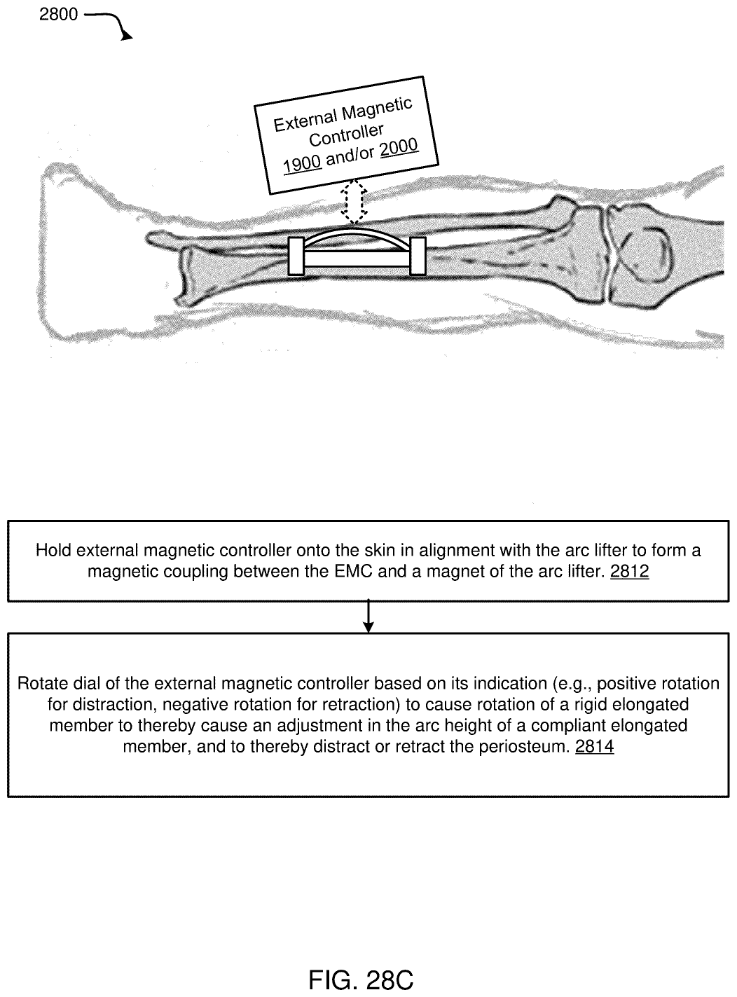

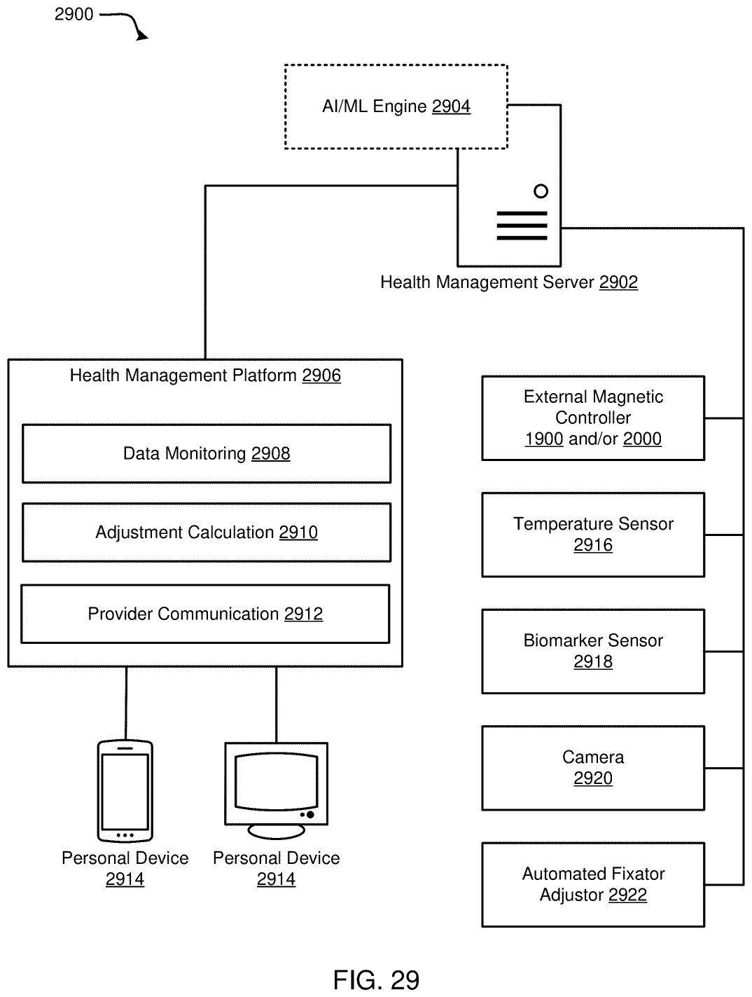

FIGS. 28A-28C are schematic block diagrams of a method 2800 for performing a periosteal distraction osteogenesis procedure. The method 2800 steps may be slightly modified to perform a similar distraction histogenesis procedure on alternative tissue types, including soft tissues. The method 2800 may be performed with an arc lifter distraction device as described herein, including at least the distraction device 100 first described in connection with FIGS. 1A-1B or the distraction device 700 first described in connection with FIGS. 7A-7B.

The method 2800 includes planning at 2802 an arc lifter position and marking the patient skin to indicate skin incision site. The method 2800 includes creating at 2804 a longitudinal skin incision and transverse periosteum incision. The method 2800 include inserting at 2806 a periosteal elevator to prepare subperiosteal tunnel. The method 2800 include inserting at 2808 an arc lifter under the patient periosteum, with the guidance of the periosteal elevator if needed. The method 2800 includes closing at 2810 the patient periosteum and skin over the top of the arc lifter. The method 2800 includes holding at 2812 the EMC 1900, 2000 onto the skin. The method 2800 includes rotating at 1910 a dial of the EMC based on its indication (e.g., positive rotation for distraction and negative rotation for retraction, or vice versa) to cause rotation of a rigid elongated member to thereby cause an adjustment in the arc height of a compliant elongated member, and to thereby distract or retract the periosteum.

FIG. 29 is a schematic block diagram of a system 2900 for remotely monitoring and controlling a distraction device such as the arc lifter distraction devices described herein. The system 2900 may be utilized in connection with any of the systems, methods, or devices described herein. The system 2900 may specifically be utilized in connection with any of the distraction devices 100, 700, 1100, 1700 described herein. The system 2900 may further be utilized in connection with an EMC 1900, 2000 as described herein, and/or an automated fixator adjustor 2922.

The system 2900 includes a health management server 2902 that processes operations for a health management platform 2906. The health management server 2902 renders a graphical user interface (GUI) that is made accessible by way of the health management platform 2906. The health management platform 2906 is made accessible to one or more personal devices 2914 by way of a computer-executed application, web browser, smart application, or other means. The health management platform 2906 may be accessed with a personal device 2914 that is in communication with a network, such as wide area network, local area network, the Internet, and so forth.

The health management server 2902 may optionally include an artificial intelligence and/or machine learning (AI/ML) engine 2904. The AI/ML engine 2904 is trained upon a set of training data. The AI/ML engine 2904 may be trained to assess data output by any of the EMC 1900, 2000, a temperature sensor 2916, a biomarker sensor 2918, a camera 2920, or an automated fixator adjustor 2922 to monitor the status of a distraction device and determine whether there is a likely issue with the patient. The AI/ML engine 2904 may further be trained on data from numerous orthopedic procedures, including osteotomy and distraction histogenesis procedures, to generate a proposed treatment protocol for adjusting a fixator with the automated fixator adjustor 2922.

The health management platform 2906 may include modules for providing information regarding data monitoring 2908, adjustment calculation 2910, provider communication 2912, and so forth. The health management platform 2906 may be rendered on an application that is run on a personal device 2914, such as a mobile phone, a tablet, or other personal computer. The health management platform 2906 may be rendered on a web browser that is accessible by way of a personal device 2914 connected to the Internet or LoRaWAN.

Patients, healthcare providers, administrators, and other users may access the health management platform 2906 with a personal device 2914 to view up-to-date data provided by any of the automated fixator adjustor 2922, temperature sensor 2916, biomarker sensor 2918, or camera 2920. Additionally, the health management platform 2906 provides current and past information regarding adjustment calculations 2910 for a fixator that is currently or was previously fixated to a patient. The adjustment calculations 2910 may be utilized to change or maintain protocols to be implemented by the automated fixator adjustor 100. The health management platform 2912 provides a means for secure bidirectional communication with patients and healthcare providers by way of the provider communication 2912 module. The health management platform 2912 additionally provides a means for secure bidirectional communication with devices such as the EMC 1900, 2000, temperature sensor 2916, biomarker sensor 2918, camera 2920, and automated fixator adjustor 2922.

The EMC 1900, 2000 may be automated to engage rotation of a magnet, which may in turn cause rotation of a magnetic portion of a rigid elongated member as described herein. The EMC 1900, 2000 may be automated to enable distraction or retraction of a compliant elongated member without manual user intervention. The EMC 1900, 2000 may include one or more processors configured to execute instructions stored in non-transitory computer readable storage medium. The processors of the EMC 1900, 2000 may receive instructions from any of the health management server 2902, a locally stored memory device with the EMC 1900, 2000, or from another source such as the personal device 2914. The EMC 1900, 2000 may automatically lift or lower the compliant elongated member of a distraction device to make real-time adjustments in accordance with a healthcare provider's protocol. A healthcare provider may amend the adjustment protocol by communicating with the health management server 2902 by way of the health management platform 2906, or through the personal device 2914.

The EMC 1900, 2000 may include one or more Bluetooth® low energy chips and/or near-field communication chips that enable the EMC 1900, 2000 to wirelessly communicate with personal devices 2914 by way of Bluetooth® or near-field communication. The EMC 1900, 2000 may further be equipped with Wi-Fi® or LoRaWAN capability that enables the EMC 1900, 2000 to wirelessly communicate with the health management server 2902 directly.

The temperature sensor 2916 may be temporarily installed within a patient to monitor the real-time internal temperature of the patient. Data output by the temperature sensor 2916 may be assessed to determine if patient tissue is inflamed or if the patient may have an infection at the surgical site or if there is enough blood flow on extremities of patient's body.

The biomarker sensor 2918 may be temporarily installed within a patient to monitor the real-time presence of certain biomarkers. The biomarker sensor 2918 may specifically be tuned to monitor the presence of biomarkers that may signal the patient currently has an infection at the surgical site. The data output by the biomarker sensor 2918 may be utilized to determine if the patient likely has an infection at the surgical site and requires additional care. The biomarker sensor 2918 may additionally measure whether the patient possesses growth factors or positive biomarkers that are known to be associated with healing.

The camera 2920 may include a standalone camera or one or more image sensors, light sources, photocells, other sensors, and other features associated with another computing device such as a mobile phone. The camera 2920 may additionally include other spectroscopy systems, including, for example, NIRS (Near InfraRed Spectroscopy) which may be utilized for measuring tissue oxygenation and to monitor the progression of perfusion to tissues. In some cases, a personal device 2914 comprising a camera communicates with the health management server 2902 by way of a computer-executed application. The health management server 2902 executes the application and may communicate directly with the camera 2920 to receive images and other data captured by the camera 2920. In some cases, the camera 2920 may be integrated with any of the systems described herein to capture up-to-date pictures of a fixator installed on a patient, which may include an external monitoring device and/or any suitable orthopedic device.

The automated fixator adjustor 2922 comprises one or more processors configured to execute instructions stored in non-transitory computer readable storage medium. The processors of the automated fixator adjustor 2922 may receive instructions from any of the health management server 2902, a locally stored memory device with the automated fixator adjustor 2922, or from another source such as the personal device 2914. The automated fixator adjustor 2922 may automatically lift or lower a compliant elongated member of a distraction device to make real-time adjustments in accordance with a healthcare provider's protocol. A healthcare provider may amend the adjustment protocol by communicating with the health management server 2902 by way of the health management platform 2906.

The automated fixator adjustor 2922 may include one or more Bluetooth® low energy chips and/or near-field communication chips that enable the automated fixator adjustor 2922 to wirelessly communicate with personal devices 2914 by way of Bluetooth® or near-field communication. The automated fixator adjustor 2922 may further be equipped with Wi-Fi® or LoRaWAN capability that enables the automated fixator adjustor 2922 to wirelessly communicate with the health management server 2902 directly.

The temperature sensor 2916, biomarker sensor 2916, and camera 2920 may include separate components that individually communicate with the health management server 2902 over a network connection. In other implementations, one or more of the temperature sensor 2916, the biomarker sensor 2918, or the camera 2920 may be integrated into the EMC 1900, 2000 and/or the automated fixator adjustor 2922.

The personal device 2914 is any personal computing device that can communicate with the health management server 2902. The personal device 2914 may include a smart phone, a tablet, a laptop, a personal computer, virtual or augmented reality device, and so forth. The personal devices 2914 may communicate with the health management server 2902 by way of a local area network (LAN) connection, a wide area network (WAN) connection, or another network connection, 4G/5G data network, or satellite signals.

The personal device 2914 may include an application installed thereon for enabling streamlined communication with the health management server 2902. The application is configured for multiple platforms, including, for example, smart phone platforms, tablet platforms, Android® operating systems, Linux® operating systems, Windows® operating systems, iOS® operating systems, Macintosh® operating systems, and so forth. The application is configured to provide secure bidirectional communication with one or more of the health management server 2902, the EMC 1900, 2000, the temperature sensor 2916, the biomarker sensor 2918, the camera 2920, or the automated fixator adjustor 2922. The application may wirelessly receive sensor information, system status information, and may further send commanding signals to sensors and system components. The application is configured to display system information through one or more means, including, for example, graphic displays, audible sounds, videos, and so forth. The application is configured to receive user input and is capable of communicating with multiple systems.

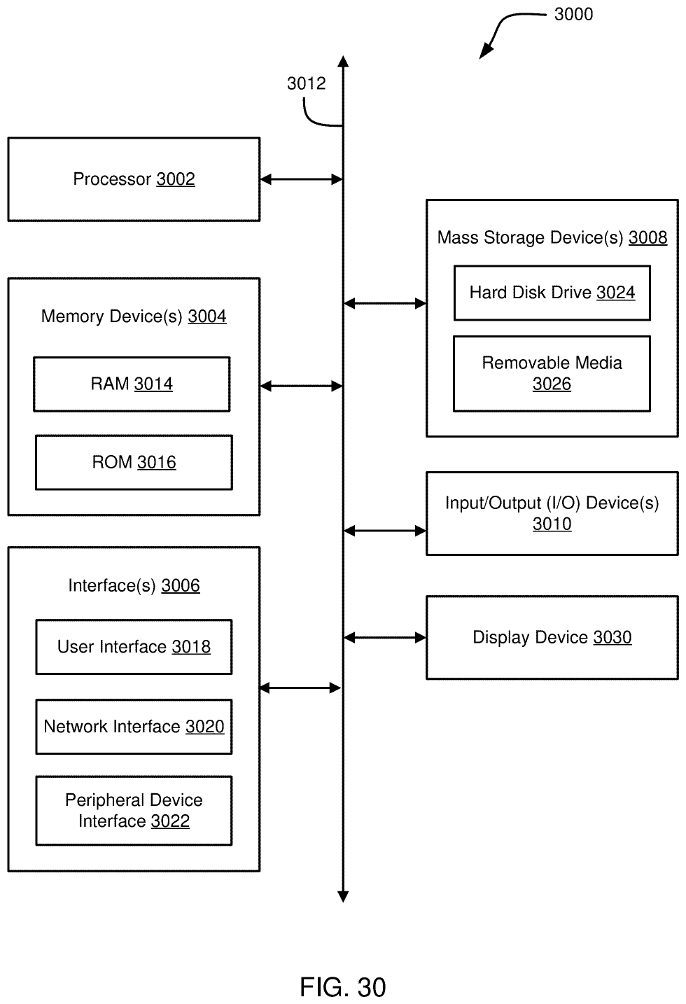

Referring now to FIG. 30, a block diagram of an example computing device 3000 is illustrated. Computing device 3000 may be used to perform various procedures, such as those discussed herein. Computing device 3000 can perform various monitoring functions as discussed herein, and can execute one or more application programs, such as the application programs or functionality described herein. Computing device 3000 can be any of a wide variety of computing devices, such as a desktop computer, in-dash computer, vehicle control system, a notebook computer, a server computer, a handheld computer, tablet computer and the like.

Computing device 3000 includes one or more processor(s) 3012, or microcontrollers, one or more memory device(s) 3004, one or more interface(s) 3006, one or more mass storage device(s) 3008, one or more Input/output (I/O) device(s) 3010, and a display device 3030 all of which are coupled to a bus 3012. Processor(s) 3012 include one or more processors or controllers that execute instructions stored in memory device(s) 3004 and/or mass storage device(s) 3008. Processor(s) 3012 may also include diverse types of computer-readable media, such as cache memory.

Memory device(s) 3004 include various computer-readable media, such as volatile memory (e.g., random access memory (RAM) 3014) and/or nonvolatile memory (e.g., read-only memory (ROM) 3016). Memory device(s) 3004 may also include rewritable ROM, such as Flash memory.

Mass storage device(s) 3008 include various computer readable media, such as magnetic tapes, magnetic disks, optical disks, solid-state memory (e.g., Flash memory), and so forth. As shown in FIG. 30, a particular mass storage device 3008 is a hard disk drive 3024. Various drives may also be included in mass storage device(s) 3008 to enable reading from and/or writing to the various computer readable media. Mass storage device(s) 3008 include removable media 3026 and/or non-removable media.

I/O device(s) 3010 include various devices that allow data and/or other information to be input to or retrieved from computing device 3000. Example I/O device(s) 3010 include cursor control devices, keyboards, keypads, microphones, monitors, touchscreen devices, or other display devices, speakers, printers, network interface cards, modems, and the like.

Display device 3030 includes any type of device capable of displaying information to one or more users of computing device 3000. Examples of display device 3030 include a monitor, display terminal, video projection device, and the like.

Interface(s) 3006 include various interfaces that allow computing device 3000 to interact with other systems, devices, or computing environments. Example interface(s) 3006 may include any number of different network interfaces 3020, such as interfaces to local area networks (LANs), wide area networks (WANs), 4G/5G data network, satellite signals, wireless networks, and the Internet. Other interface(s) include user interface 3018 and peripheral device interface 3022. The interface(s) 3006 may also include one or more user interface elements 3018. The interface(s) 3006 may also include one or more peripheral interfaces such as interfaces for printers, pointing devices (mice, track pad, or any suitable user interface now known to those of ordinary skill in the field, or later discovered), keyboards, and the like.

Bus 3012 allows processor(s) 3012, memory device(s) 3004, interface(s) 3006, mass storage device(s) 3008, and I/O device(s) 3010 to communicate with one another, as well as other devices or components coupled to bus 3012. Bus 3012 represents one or more of several types of bus structures, such as a system bus, PCI bus, IEEE bus, USB bus, and so forth.

For purposes of illustration, programs and other executable program components are shown herein as discrete blocks, although it is understood that such programs and components may reside at various times in different storage components of computing device 1800 and are executed by processor(s) 3012. Alternatively, the systems and procedures described herein can be implemented in hardware, or a combination of hardware, software, and/or firmware. For example, one or more application specific integrated circuits (ASICs) can be programmed to conduct one or more of the systems and procedures described herein. As used herein, the terms “module” or “component” are intended to convey the implementation apparatus for accomplishing a process, such as by hardware, or a combination of hardware, software, and/or firmware, for the purposes of performing all or parts of operations disclosed herein. The terms “module” or “component” are intended to convey independent in how the modules, components, or their functionality or hardware may be implemented in different embodiments.

### EXAMPLES

The following examples pertain to further embodiments.

Example 1 is a device for performing a distraction histogenesis procedure. The device includes a rigid elongated member comprising external threading. The device includes a compliant elongated member, wherein a first end of the compliant elongated member is coupled to the rigid elongated member. The device includes a fastener comprising a hole defined by a sidewall, wherein the fastener comprises internal threading on the sidewall, and wherein the fastener couples a second end of the compliant elongated member to the rigid elongated member. The device is such that rotation of the rigid elongated member causes movement of the fastener.

Example 2 is a device as in Example 1, further comprising a swivel joint, wherein the swivel joint couples the first end of the compliant elongated member to the rigid elongated member.

Example 3 is a device as in any of Examples 1-2, wherein the external threading of the rigid elongated member corresponds with the internal threading of the fastener.

Example 4 is a device as in any of Examples 1-3, wherein the fastener is screwed onto the rigid elongated member such that the fastener move along a longitudinal axis of the rigid elongated member in response to the rotation of the rigid elongated member.

Example 5 is a device as in any of Examples 1-4, wherein the second end of the compliant elongated member is attached to the fastener such that the second end of the compliant elongated member along the longitudinal axis of the rigid elongated member in response to the fastener moving along the longitudinal axis of the rigid elongated member.

Example 6 is a device as in any of Examples 1-5, wherein the second end of the compliant elongated member is located opposite to the first end of the compliant elongated member; and wherein the first end of the compliant elongated member and the swivel joint are located substantially adjacent to a first end of the rigid elongated member.

Example 7 is a device as in any of Examples 1-6, wherein a second end of the rigid elongated member is located opposite to the first end of the rigid elongated member; and wherein the fastener and the second end of the compliant elongated member are located substantially adjacent to the second end of the rigid elongated member when the compliant elongated member is in a maximum relaxed state.

Example 8 is a device as in any of Examples 1-7, wherein the fastener and the second end of the compliant elongated member move toward the first end of the rigid elongated member in response to the rotation of the rigid elongated member; wherein the compliant elongated member is in a maximum bent state when the fastener is located substantially adjacent to the swivel joint.

Example 9 is a device as in any of Examples 1-8, wherein rotation of the rigid elongated member in a first rotational direction causes the fastener to move toward the swivel joint; and wherein rotation of the rigid elongated member in a second rotational direction causes the fastener to move away from the swivel joint.

Example 10 is a device as in any of Examples 1-9, wherein the rotation of the rigid elongated member in a first rotational direction causes the fastener to move toward the swivel joint; wherein the swivel joint swivels in response to the fastener moving toward the swivel joint; and wherein the compliant elongated member bends in response to the fastener moving toward the swivel join.

Example 11 is a device as in any of Examples 1-10, further comprising a drive head attached to the rigid elongated member at a first end of the rigid elongated member.

Example 12 is a device as in any of Examples 1-11, wherein the drive head comprises an impression configured to receive a drill head; wherein rotation of the drill head causes rotation of the rigid elongated member when the drill head is disposed within the impression of the drive head; and wherein rotation of the rigid elongated member in a first rotational direction causes the fastener to move toward the swivel join and further causes the compliant elongated member to bend away from the rigid elongated member.

Example 13 is a device as in any of Examples 1-12, wherein the device is configured to be disposed in between a periosteum layer and an outer surface of a bone.

Example 14 is a device as in any of Examples 1-13, wherein the device is configured to be disposed in between a periosteum layer and an outer surface of a bone to perform a periosteal distraction osteogenesis procedure to initiate regeneration of the bone.

Example 15 is a device as in any of Examples 1-14, wherein the device is disposed in between a periosteum layer and an outer surface of a bone during the distraction osteogenesis procedure; wherein the rotation of the rigid elongated member in a first rotational direction causes the fastener to move toward the swivel joint, and further causes the compliant elongated member to bend away from the rigid elongated member; and wherein the compliant elongated member bending away from the rigid elongated member causes the periosteum to pull away from the outer surface of the bone.

Example 16 is a device as in any of Examples 1-15, wherein the rotation of the rigid elongated member in a second rotational direction causes the fastener to move away from the swivel joint, and further causes the compliant elongated member to straighten toward the rigid elongated member; and wherein the compliant elongated member straightening toward the rigid elongated member causes the periosteum to relax down toward the outer surface of the bone.

Example 17 is a device as in any of Examples 1-16, wherein the distraction osteogenesis procedure is a periosteal distraction osteogenesis procedure; and wherein, during the periosteal distraction osteogenesis procedure, the rotation of the rigid elongated member causes distraction or retraction of a periosteum of a patient.

Example 18 is a device as in any of Examples 1-17, wherein the compliant elongated member comprises a first compliant elongated member and a second compliant elongated member; wherein a longitudinal axis of the first compliant elongated member is substantially parallel to a longitudinal axis of the rigid elongated member; and wherein a longitudinal axis of the second compliant elongated member is substantially parallel to the longitudinal axis of the rigid elongated member.

Example 19 is a device as in any of Examples 1-18, wherein the rigid elongated member is disposed in between the first compliant elongated member and the second compliant elongated member such that the first compliant elongated member is disposed adjacent to a first side of the rigid elongated member, and the second compliant elongated member is disposed adjacent to a second side of the rigid elongated member.

Example 20 is a device as in any of Examples 1-19, further comprising a nut attached to the rigid elongated member, wherein rotation of the nut causes rotation of the rigid elongated member.

Example 21 is a device for performing a distraction histogenesis procedure. The device includes a rigid elongated member comprising a magnetic portion and a threaded portion. The device includes an adjustment coupler comprising a hollow interior defined by a sidewall, wherein the adjustment coupler comprises internal threading attached to the sidewall. The device includes a compliant elongated member coupled to each of the adjustment coupler and the rigid elongated member. The device is such that the internal threading of the adjustment coupler corresponds with the threaded portion of the rigid elongated member. The device is such that rotation of the rigid elongated member causes the threaded portion to screw into or out of the sidewall of the adjustment coupler.

Example 22 is a device as in Example 21, wherein the adjustment coupler couples the compliant elongated member to the rigid elongated member at a first end of the compliant elongated member; and wherein the device further comprises a stationary coupler that couples the compliant elongated member to the rigid elongated member at a second end of the compliant elongated member.

Example 23 is a device as in any of Examples 21-22, wherein the rotation of the rigid elongated member causes the threaded portion to screw into the sidewall of the adjustment coupler when the rigid elongated member is rotated in a tightening direction; and wherein the compliant elongated member distracts away from a longitudinal axis of the rigid elongated member in response to the threaded portion screwing into the sidewall of the adjustment coupler.

Example 24 is a device as in any of Examples 21-23, wherein the rotation of the rigid elongated member causes the threaded portion to screw out of the sidewall of the adjustment coupler when the rigid elongated member is rotated in a loosening direction; and wherein the compliant elongated member retracts toward a longitudinal axis of the rigid elongated member in response to the threaded portion screwing out of the sidewall of the adjustment coupler.

Example 25 is a device as in any of Examples 21-24, wherein the magnetic portion of the rigid elongated member comprises an elongated magnet, and wherein the elongated magnet comprises at least two poles extending a length of the elongated magnet.

Example 26 is a device as in any of Examples 21-25, wherein the elongated magnet forms a magnetic coupling with a corresponding magnet or electromagnet of an external magnetic controller; and wherein rotation of the corresponding magnet causes corresponding rotation of the elongated magnet.

Example 27 is a device as in any of Examples 21-26, wherein corresponding rotation of the elongated magnet in a tightening direction causes corresponding rotation of the rigid elongated member in the tightening direction; wherein the corresponding rotation of the rigid elongated member in the tightening direction causes the threaded portion to screw into the sidewall of the adjustment coupler; and wherein the threaded portion screwing into the sidewall of the adjustment coupler causes the compliant elongated member to distract away from a longitudinal axis of the rigid elongated member.

Example 28 is a device as in any of Examples 21-27, wherein corresponding rotation of the elongated magnet in a loosening direction causes corresponding rotation of the rigid elongated member in the loosening direction; wherein the corresponding rotation of the rigid elongated member in the loosening direction causes the threaded portion to screw out of the sidewall of the adjustment coupler; and wherein the threaded portion screwing out of the sidewall of the adjustment coupler causes the compliant elongated member to retract toward a longitudinal axis of the rigid elongated member.

Example 29 is a device as in any of Examples 21-28, further comprising a pivot head coupled to the adjustment coupler by way of a pivot fastener; wherein the compliant elongated member is coupled to the adjustment coupler by way of the pivot head.

Example 30 is a device as in any of Examples 21-29, wherein the adjustment coupler couples the compliant elongated member to the rigid elongated member at a first end of the compliant elongated member; wherein the device further comprises a stationary coupler that couples the compliant elongated member to the rigid elongated member at a second end of the compliant elongated member; wherein the device further comprises a first pivot head coupled to the adjustment coupler by way of a first pivot fastener; wherein the device further comprises a second pivot head coupled to the stationary coupler by way of a second pivot fastener; wherein the compliant elongated member is coupled to the adjustment coupler by way of the first pivot head; and wherein the compliant elongated member is coupled to the stationary coupler by way of the second pivot head.

Example 31 is a device as in any of Examples 21-30, wherein the first pivot head and the second pivot head enable the compliant elongated member to distract away from a longitudinal axis of the rigid elongated member in an arc configuration.

Example 32 is a device as in any of Examples 21-31, wherein the compliant elongated member comprises a first end and a second end that is opposite to the first end; wherein a distance between the first end and the second end of the compliant elongated member is an arc chord of the compliant elongated member; and wherein the arc chord is adjustable by screwing the threaded portion into or out of the sidewall of the adjustment coupler.

Example 33 is a device as in any of Examples 21-32, wherein the device is configured to be disposed in between a periosteum layer and an outer surface of a bone.

Example 34 is a device as in any of Examples 21-33, wherein the device is configured to be disposed in between a periosteum layer and an outer surface of a bone to perform a periosteal distraction osteogenesis procedure to initiate regeneration of the bone by distracting the periosteum layer away from the outer surface of the bone.

Example 35 is a device as in any of Examples 21-34, wherein the device is disposed in between the periosteum layer and the outer surface of the bone during the periosteal distraction osteogenesis procedure; wherein rotation of the rigid elongated member in a tightening direction causes the threaded portion to screw into the sidewall of the adjustment coupler and further causes the compliant elongated member to distract away from the rigid elongated member; and wherein the compliant elongated member distracting away from the rigid elongated member causes the periosteum to stretch and distract away from the outer surface of the bone.

Example 36 is a device as in any of Examples 21-35, wherein the device is disposed in between the periosteum layer and the outer surface of the bone during the periosteal distraction osteogenesis procedure; wherein rotation of the rigid elongated member in a loosening direction causes the threaded portion to screw out of the sidewall of the adjustment coupler and further causes the compliant elongated member to retract toward the rigid elongated member; and wherein the compliant elongated member retracting toward the rigid elongated member allows the periosteum to relax toward the outer surface of the bone.

Example 37 is a device as in any of Examples 21-36, wherein the distraction histogenesis procedure is a periosteal distraction osteogenesis procedure; and wherein, during the periosteal distraction osteogenesis procedure, the rotation of the rigid elongated member causes distraction or retraction of a periosteum of a patient.

Example 38 is a device as in any of Examples 21-37, wherein the magnetic portion of the rigid elongated member comprises an elongated magnet; wherein the rigid elongated member further comprises a housing disposed around the elongated magnet; and wherein the housing is attached to the threaded portion of the rigid elongated member.

Example 39 is a device as in any of Examples 21-38, wherein the device is installed within a patient during the distraction histogenesis procedure such that a skin barrier of the patient is closed over the device; wherein the magnetic portion of the rigid elongated member forms a magnetic coupling with a corresponding magnet of an external magnetic controller when the device is installed within the patient.

Example 40 is a device as in any of Examples 21-39, wherein, in response to establishing the magnetic coupling between the external magnetic controller and the magnetic portion of the rigid elongated member, the corresponding magnet is rotated in a first direction to cause corresponding rotation of the rigid elongated member; and wherein the corresponding rotation of the rigid elongated member causes adjustment to a length of an arc chord formed between a first end of the compliant elongated member and a second end of the compliant elongated member.

The foregoing description has been presented for purposes of illustration. It is not exhaustive and does not limit the invention to the precise forms or embodiments disclosed. Modifications and adaptations will be apparent to those skilled in the art from consideration of the specification and practice of the disclosed embodiments. For example, components described herein may be removed and other components added without departing from the scope or spirit of the embodiments disclosed herein or the appended claims.

Other embodiments will be apparent to those skilled in the art from consideration of the specification and practice of the disclosure disclosed herein. It is intended that the specification and examples be considered as exemplary only, with a true scope and spirit of the invention being indicated by the following claims.

What is claimed is:

1. A device for performing a distraction histogenesis procedure, the device comprising:
a rigid elongated member comprising a magnetic portion and a threaded portion;
an adjustment coupler comprising a hollow interior defined by a sidewall, wherein the adjustment coupler comprises internal threading attached to the sidewall; and
a compliant elongated member coupled to each of the adjustment coupler and the rigid elongated member;
wherein the internal threading of the adjustment coupler corresponds with the threaded portion of the rigid elongated member; and
wherein rotation of the rigid elongated member causes the threaded portion to screw into or out of the sidewall of the adjustment coupler.

2. The device of claim 1, wherein the adjustment coupler couples the compliant elongated member to the rigid elongated member at a first end of the compliant elongated member; and
wherein the device further comprises a stationary coupler that couples the compliant elongated member to the rigid elongated member at a second end of the compliant elongated member.

3. The device of claim 1, wherein the rotation of the rigid elongated member causes the threaded portion to screw into the sidewall of the adjustment coupler when the rigid elongated member is rotated in a tightening direction; and
wherein the compliant elongated member bends away from a longitudinal axis of the rigid elongated member in response to the threaded portion screwing into the sidewall of the adjustment coupler.

4. The device of claim 1, wherein the rotation of the rigid elongated member causes the threaded portion to screw out of the sidewall of the adjustment coupler when the rigid elongated member is rotated in a loosening direction; and
wherein the compliant elongated member retracts toward a longitudinal axis of the rigid elongated member in response to the threaded portion screwing out of the sidewall of the adjustment coupler.

5. The device of claim 1, wherein the magnetic portion of the rigid elongated member comprises an elongated magnet, and wherein the elongated magnet comprises at least two poles extending a length of the elongated magnet.

6. The device of claim 5, wherein the elongated magnet forms a magnetic coupling with a corresponding magnet of an external magnetic controller, wherein the corresponding magnet of the external magnetic controller comprises one or more of a magnet or an electromagnet; and
wherein rotation of the corresponding magnet causes corresponding rotation of the elongated magnet.

7. The device of claim 6, wherein corresponding rotation of the elongated magnet in a tightening direction causes corresponding rotation of the rigid elongated member in the tightening direction;
wherein the corresponding rotation of the rigid elongated member in the tightening direction causes the threaded portion to screw into the sidewall of the adjustment coupler; and
wherein the threaded portion screwing into the sidewall of the adjustment coupler causes the compliant elongated member to distract away from a longitudinal axis of the rigid elongated member.

8. The device of claim 6, wherein corresponding rotation of the elongated magnet in a loosening direction causes corresponding rotation of the rigid elongated member in the loosening direction;
wherein the corresponding rotation of the rigid elongated member in the loosening direction causes the threaded portion to screw out of the sidewall of the adjustment coupler; and
wherein the threaded portion screwing out of the sidewall of the adjustment coupler causes the compliant elongated member to retract toward a longitudinal axis of the rigid elongated member.

9. The device of claim 1, further comprising a pivot head coupled to the adjustment coupler by way of a pivot fastener;
wherein the compliant elongated member is coupled to the adjustment coupler by way of the pivot head.

10. The device of claim 1, wherein the adjustment coupler couples the compliant elongated member to the rigid elongated member at a first end of the compliant elongated member;
wherein the device further comprises a stationary coupler that couples the compliant elongated member to the rigid elongated member at a second end of the compliant elongated member;
wherein the device further comprises a first pivot head coupled to the adjustment coupler by way of a first pivot fastener;
wherein the device further comprises a second pivot head coupled to the stationary coupler by way of a second pivot fastener;
wherein the compliant elongated member is coupled to the adjustment coupler by way of the first pivot head; and
wherein the compliant elongated member is coupled to the stationary coupler by way of the second pivot head.

11. The device of claim 10, wherein the first pivot head and the second pivot head enable the compliant elongated member to distract away from a longitudinal axis of the rigid elongated member in an arc configuration.

12. The device of claim 1, wherein the compliant elongated member comprises a first end and a second end that is opposite to the first end;
wherein a distance between the first end and the second end of the compliant elongated member is an arc chord of the compliant elongated member; and
wherein the arc chord is adjustable by screwing the threaded portion into or out of the sidewall of the adjustment coupler.

13. The device of claim 1, wherein the device is configured to be disposed in between a periosteum layer and an outer surface of a bone.

14. The device of claim 1, wherein the device is configured to be disposed in between a periosteum layer and an outer surface of a bone to perform a periosteal distraction procedure to improve perfusion and initiate regeneration of the bone or soft tissue by stretching and distracting the periosteum layer away from the outer surface of the bone.

15. The device of claim 14, wherein the device is disposed in between the periosteum layer and the outer surface of the bone during the periosteal distraction procedure;
wherein rotation of the rigid elongated member in a tightening direction causes the threaded portion to screw into the sidewall of the adjustment coupler and further causes the compliant elongated member to distract away from the rigid elongated member; and
wherein the compliant elongated member distracting away from the rigid elongated member causes the periosteum to stretch and distract away from the outer surface of the bone.

16. The device of claim 14, wherein the device is disposed in between the periosteum layer and the outer surface of the bone during the periosteal distraction procedure;
wherein rotation of the rigid elongated member in a loosening direction causes the threaded portion to screw out of the sidewall of the adjustment coupler and further causes the compliant elongated member to retract toward the rigid elongated member; and
wherein the compliant elongated member retracting toward the rigid elongated member allows the periosteum to relax toward the outer surface of the bone.

17. The device of claim 1, wherein the distraction histogenesis procedure is a periosteal distraction procedure; and
wherein, during the periosteal distraction procedure, the rotation of the rigid elongated member causes distraction or retraction of a periosteum of a patient.

18. The device of claim 1, wherein the magnetic portion of the rigid elongated member comprises an elongated magnet;
wherein the rigid elongated member further comprises a housing disposed around the elongated magnet; and
wherein the housing is attached to the threaded portion of the rigid elongated member.

19. The device of claim 1, wherein the device is installed within a patient during the distraction histogenesis procedure such that a skin barrier of the patient is closed over the device;
wherein the magnetic portion of the rigid elongated member forms a magnetic coupling with a corresponding magnet of an external magnetic controller when the device is installed within the patient.

20. The device of claim 19, wherein, in response to establishing the magnetic coupling between the external magnetic controller and the magnetic portion of the rigid elongated member, the corresponding magnet is rotated in a first direction to cause corresponding rotation of the rigid elongated member; and
wherein the corresponding rotation of the rigid elongated member causes adjustment to a length of an arc chord formed between a first end of the compliant elongated member and a second end of the compliant elongated member.
